(19) United States
(12) Patent Application Publication
(10) Pub. No.: US 2025/0154130 A1
(43) Pub. Date: May 15, 2025

(54) NOVEL THR BETA ANALOGS AND USES THEREOF

(71) Applicant: MADRIGAL PHARMACEUTICALS, INC., West Conshohocken, PA (US)

(72) Inventors: JAMES K. HENNAN, Harleysville, PA (US); PASQUALE N. CONFALONE, Wilmington, DE (US)

(21) Appl. No.: 18/837,378

(22) PCT Filed: Feb. 10, 2023
(86) PCT No.: PCT/US2023/062351

§ 371 Date: Aug. 9, 2024

Related U.S. Application Data
(60) Provisional application No. 63/419,790, filed on Oct. 27, 2022.
(60) Provisional application No. 63/308,709, filed on Feb. 10, 2022.

(51) Int. Cl. C07D 403/12; A61K 31/513

(52) U.S. Cl. CPC C07D 403/12; A61K 31/513

(57) ABSTRACT

Described herein are compounds of formula I:

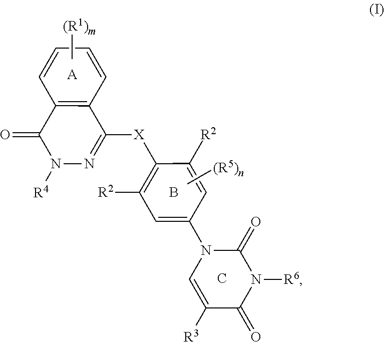

and pharmaceutically acceptable salts, solvates, and stereoisomers thereof, and their uses as thyroid hormone receptor (THR) β activators.

### RELATED APPLICATIONS

This application claims priority to and the benefit of U.S. Provisional Application No. 63/308,709, filed on Feb. 10, 2022, and U.S. Provisional Application No. 63/419,790, filed on Oct. 27, 2022, the contents of which are incorporated herein by reference.

### INCORPORATION-BY-REFERENCE OF SEQUENCE LISTING

The contents of the file named “MDRI-028_001WO_SeqList_ST26.txt”, which was created Feb. 7, 2023, and is 1.87 KB in size are hereby incorporated by reference in their entirety.

### BACKGROUND

Thyroid hormones are critical for normal growth and development and for maintaining metabolic homeostasis (Paul M. Yen, Physiological reviews, Vol. 81(3): pp. 1097-1126 (2001)). Circulating levels of thyroid hormones are tightly regulated by feedback mechanisms in the hypothalamus/pituitary/thyroid (HPT) axis. Thyroid dysfunction leading to hypothyroidism or hyperthyroidism clearly demonstrates that thyroid hormones exert profound effects on cardiac function, body weight, metabolism, metabolic rate, body temperature, cholesterol, bone, muscle, and behavior.

The biological activity of thyroid hormones is mediated by thyroid hormone receptors (TRs or THRs) (M. A. Lazar, Endocrine Reviews, Vol. 14: pp. 348-399 (1993)). TRs belong to the superfamily known as nuclear receptors. TRs form heterodimers with the retinoid receptor that act as ligand-inducible transcription factors. TRs have a ligand binding domain, a DNA binding domain, and an amino terminal domain, and regulate gene expression through interactions with DNA response elements and with various nuclear co-activators and co-repressors. The thyroid hormone receptors are derived from two separate genes, α and β. These distinct gene products produce multiple forms of their respective receptors through differential RNA processing. The major thyroid receptor isoforms are α1, α2, β1 and β2. Thyroid hormone receptors α1, β1 and β2 bind thyroid hormone. It has been shown that the thyroid hormone receptor subtypes can differ in their contribution to particular biological responses. Recent studies suggest that TRβ1 plays an important role in regulating TRH (thyrotropin releasing hormone) and on regulating thyroid hormone actions in the liver. TRβ2 plays an important role in the regulation of TSH (thyroid stimulating hormone) (Abel et. al., J. Clin. Invest., Vol 104: pp. 291-300 (1999)). TRβ1 plays an important role in regulating heart rate (B. Gloss et. al. Endocrinology, Vol. 142: pp. 544-550 (2001); C. Johansson et. al., Am. J. Physiol., Vol. 275: pp. R640-R646 (1998)).

Efforts have been made to synthesize thyroid hormone analogs which exhibit increased thyroid hormone receptor beta selectivity and/or tissue selective action. Such thyroid hormone mimetics may yield desirable reductions in body weight, lipids, cholesterol, and lipoproteins, with reduced impact on cardiovascular function or normal function of the hypothalamus/pituitary/thyroid axis (see, e.g., Joharapurkar et al., J. Med. Chem., 2012, 55 (12), pp 5649-5675). The development of thyroid hormone analogs which avoid the undesirable effects of hyperthyroidism and hypothyroidism while maintaining the beneficial effects of thyroid hormones would open new avenues of treatment for patients with metabolic disease such as obesity, hyperlipidemia, hypercholesterolemia, diabetes and other disorders and diseases such as liver steatosis and NASH, atherosclerosis, cardiovascular diseases, hypothyroidism, thyroid cancer, thyroid diseases, resistance to thyroid hormone and related disorders and diseases.

### SUMMARY

In some aspects, the present disclosure provides compounds of Formula I:

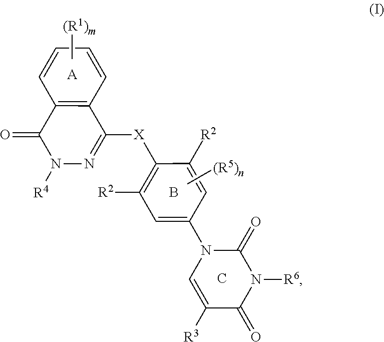

or pharmaceutically acceptable salts, solvates, or stereoisomers thereof, wherein:
each R¹ is independently halogen, C₁₋₆ alkyl, C₂₋₆ alkenyl, C₂₋₆ alkynyl, C₁₋₆ alkoxy, C₃₋₁₀ carbocyclyl, or C₆₋₁₄ aryl;
each R² is independently halogen, C₁₋₆ alkyl, C₂₋₆ alkenyl, or C₂₋₆ alkynyl;
R³ is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
each occurrence of R⁴ and R⁶ is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
each R⁵ is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
m is an integer selected from 0 to 4;
n is an integer selected from 0 to 2;
X is selected from —C(R⁷)₂—, —O—, —NR⁸—, and —S—;
each R⁷ is independently hydrogen, halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u}; and
R⁸ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
wherein:
each R^{u} is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, and heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
two R^{u} are taken together to form an oxo; or
two R^{u}, together with the one or more intervening atoms, form a C₆₋₁₄ aryl, 5- to 14-membered heteroaryl, C₃₋₁₀ carbocyclyl, or 3- to 10-membered heterocyclyl;
each R^{a} is independently C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
each R^{b} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl; and
each R^{c} and R^{d} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
or R^{c}, R^{d}, together with the nitrogen atom to which they are attached, form a 3- to 10-membered heterocyclyl optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In some aspects, the present disclosure provides a method of preparing a compound described herein.

In some aspects, the present disclosure features a pharmaceutical composition comprising any compound described herein and a pharmaceutically acceptable excipient.

In some aspects, the present disclosure features a method of treating diseases, disorders, or conditions, comprising administering to a subject in need thereof any compound described herein in a pharmaceutical composition.

In some aspects, the present disclosure features any compound described herein in a pharmaceutical composition for use for treating diseases, disorders, or conditions, comprising administering to a subject in need thereof.

In some aspects, the present disclosure features use of any compound described herein in a pharmaceutical composition in the manufacture of a medicament for treating diseases, disorders, or conditions, comprising administering to a subject in need thereof.

In some aspects, the present disclosure features a method of activating thyroid hormone receptor (THR) R in a patient or a biological sample, the method comprising contacting the patient or biological sample with the compound described herein in a pharmaceutical composition.

In some aspects, the present disclosure features any compound described herein in a pharmaceutical composition for use in activating thyroid hormone receptor (THR) R in a patient or a biological sample.

In some aspects, the present disclosure features use of the any compound described herein in a pharmaceutical composition in the manufacture of a medicament for activating thyroid hormone receptor (THR) R in a patient or a biological sample.

In some aspects, the present disclosure features a method of treating a liver disease or disorder, or a lipid disease or disorder, the method comprising administering to a subject in need thereof any compound described herein in a pharmaceutical composition.

In some aspects, the present disclosure features any compound described herein in a pharmaceutical composition for use in treating a liver disease or disorder, or a lipid disease or disorder.

In some aspects, the present disclosure features use of the any compound described herein in a pharmaceutical composition in the manufacture of a medicament for treating a liver disease or disorder, or a lipid disease or disorder.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

### Compounds of the Present Disclosure

In some aspects, the present disclosure provides compounds of Formula I:

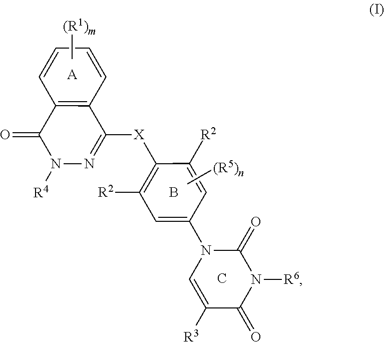

or pharmaceutically acceptable salts, solvates, or stereoisomers thereof, wherein:
each R¹ is independently halogen, C₁₋₆ alkyl, C₂₋₆ alkenyl, C₂₋₆ alkynyl, C₁₋₆ alkoxy, C₃₋₁₀ carbocyclyl, or C₆₋₁₄ aryl;
each R² is independently halogen, C₁₋₆ alkyl, C₂₋₆ alkenyl, or C₂₋₆ alkynyl; R³ is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
each occurrence of R⁴ and R⁶ is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
each R⁵ is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
m is an integer selected from 0 to 4;
n is an integer selected from 0 to 2;
X is selected from —C(R⁷)₂—, —O—, —NR⁸—, and —S—;
each R⁷ is independently hydrogen, halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u}; and
R⁸ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
wherein:
each R^{u} is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, and heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
two R^{u} are taken together to form an oxo; or
two R^{u}, together with the one or more intervening atoms, form a C₆₋₁₄ aryl, 5- to 14-membered heteroaryl, C₃₋₁₀ carbocyclyl, or 3- to 10-membered heterocyclyl;
each R^{a} is independently C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
each R^{b} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl; and each R^{c} and R^{d} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
or R^{c}, R^{d}, together with the nitrogen atom to which they are attached, form a 3- to 10-membered heterocyclyl optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In some aspects, the present disclosure provides compounds of Formula I, or pharmaceutically acceptable salts, solvates, or stereoisomers thereof, wherein:
each R¹ is independently halogen, C₁₋₆ alkyl, C₂₋₆ alkenyl, C₂₋₆ alkynyl, C₃₋₁₀ carbocyclyl, or C₆₋₁₄ aryl;
each R² is independently halogen, C₁₋₆ alkyl, C₂₋₆ alkenyl, or C₂₋₆ alkynyl;
R³ is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
each occurrence of R⁴ and R⁶ is independently C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
each R⁵ is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
m is an integer selected from 0 to 4;
n is an integer selected from 0 to 2;
X is selected from —C(R⁷)₂—, —O—, —NR⁸—, and —S—;
each R⁷ is independently hydrogen, halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u}; and
R⁸ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
wherein:
each R^{u} is independently halogen, —CN, —NO₂, C₁₋₆ alkyl, C₁₋₆haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, and heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
two R^{u} are taken together to form an oxo; or
two R^{u}, together with the one or more intervening atoms, form a C₆₋₁₄ aryl, 5- to 14-membered heteroaryl, C₃₋₁₀ carbocyclyl, or 3- to 10-membered heterocyclyl;
each R^{a} is independently C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
each R^{b} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl; and
each R^{c} and R^{d} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
or R^{c}, R^{d}, together with the nitrogen atom to which they are attached, form a 3- to 10-membered heterocyclyl optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In some aspects, the present disclosure provides compounds of Formula I, or pharmaceutically acceptable salts, solvates, or stereoisomers thereof, wherein:
each R¹ is independently halogen, C₁₋₆ alkyl, or C₁₋₆ alkoxy;
each R² is independently halogen;
R³ is —CN, —C(═O)OR^{b}, or —C(═O)NR^{c}R^{d};
each occurrence of R⁴ and R⁶ is independently hydrogen;
m is an integer selected from 0, 1, or 2;
n is 0;
X is —O—;
each R^{b} is independently hydrogen;
each R and R^{d} is independently hydrogen.

In certain embodiments, each R¹ is independently halogen, C₁₋₆ alkyl, C₁₋₆ alkoxy, or C₆₋₁₄ aryl.

In certain embodiments, at least one R¹ is halogen (e.g., F, Cl, or Br).

In certain embodiments, at least one R¹ is F or C₁.

In certain embodiments, at least one R¹ is F.

In certain embodiments, at least one R¹ is Cl.

In certain embodiments, at least one R¹ is C₁₋₆ alkyl (e.g., methyl, ethyl, or propyl).

In certain embodiments, at least one R¹ is methyl.

In certain embodiments, at least one R¹ is C₁₋₆ alkoxy (e.g., methoxy, ethoxy, or propoxy).

In certain embodiments, at least one R¹ is methoxy.

In certain embodiments, at least one R¹ is C₆₋₁₄ aryl (e.g., phenyl).

In certain embodiments, m is 0, 1, or 2.

In certain embodiments, m is 0. In certain embodiments, m is 1. In certain embodiments, m is 2.

In certain embodiments, each R² independently is halogen, C₁₋₆ alkyl, C₂₋₆ alkenyl, or C₂₋₆ alkynyl.

In certain embodiments, each R² independently is halogen (e.g., F, Cl, or Br).

In certain embodiments, each R² is chloride.

In certain embodiments, each R² independently is C₁₋₆ alkyl (e.g., methyl, ethyl, or propyl).

In certain embodiments, each R² is methyl.

In certain embodiments, each R² independently is C₂₋₆ alkenyl (e.g., ethynyl, propenyl, butenyl, pentenyl, or hexenyl).

In certain embodiments, each R² independently is C₂₋₆ alkynyl (e.g., ethynyl, propynyl, butynyl, pentynyl, or hexynyl).

In certain embodiments, R³ is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, R³ is halogen (e.g., F, Cl, or Br).

In certain embodiments, R³ is C₁₋₆ alkyl (e.g., methyl, ethyl, or propyl), wherein the alkyl is optionally substituted with one or more R^{u}.

In certain embodiments, R³ is C₂₋₆ alkenyl (e.g., ethynyl, propenyl, butenyl, pentenyl, or hexenyl), wherein the alkenyl is optionally substituted with one or more R^{u}.

In certain embodiments, R³ is C₂₋₆ alkynyl (e.g., ethynyl, propynyl, butynyl, pentynyl, or hexynyl), wherein the alkynyl is optionally substituted with one or more R^{u}.

In certain embodiments, R³ is C₁₋₆haloalkyl (e.g., C₁₋₆ alkyl groups substituted with one or more halogen atom), wherein the haloalkyl is optionally substituted with one or more R^{u}.

In certain embodiments, R³ is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, R³ is C₁₋₆ aminoalkyl (e.g., C₁₋₆ alkyl groups substituted with one or more amino groups), wherein the aminoalkyl is optionally substituted with one or more R^{u}.

In certain embodiments, R³ is C₆₋₁₄ aryl (e.g., phenyl or naphthyl), wherein the aryl is optionally substituted with one or more R^{u}.

In certain embodiments, R³ is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, R³ is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, R³ is or 3- to 10-membered heterocyclyl (e.g., tetrahydrofuranyl, piperidinyl, or tetrahydropyranyl), wherein the heterocyclyl is optionally substituted with one or more R^{u}.

In certain embodiments, R³ is —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d} (e.g., R^{a} is methyl or ethyl, R^{b} is H, R^{c} is H, and R^{d} is H).

In certain embodiments, R³ is —CN, —C(═O)OR^{b}, or —C(═O)NR^{c}R^{d}.

In certain embodiments, R³ is —CN.

In certain embodiments, each R^{c} and R^{c} is H.

In certain embodiments, R³ is —C(═O)NH₂.

In certain embodiments, R^{b} is H.

In certain embodiments, R³ is —C(═O)OH.

In certain embodiments, R⁴ is hydrogen.

In certain embodiments, n is 0, 1, or 2.

In certain embodiments, n is 0.

In certain embodiments, R⁶ is hydrogen.

In certain embodiments, R⁴ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, R⁴ is C₁₋₆ alkyl (e.g., methyl, ethyl, or propyl), wherein the alkyl is optionally substituted with one or more R^{u}.

In certain embodiments, R⁴ is C₂₋₆ alkenyl (e.g., ethynyl, propenyl, butenyl, pentenyl, or hexenyl), wherein the alkenyl is optionally substituted with one or more R^{u}.

In certain embodiments, R⁴ is C₂₋₆ alkynyl (e.g., ethynyl, propynyl, butynyl, pentynyl, or hexynyl), wherein the alkynyl is optionally substituted with one or more R^{u}.

In certain embodiments, R⁴ is C₁₋₆haloalkyl (e.g., C₁₋₆ alkyl groups substituted with one or more halogen atom), wherein the haloalkyl is optionally substituted with one or more R^{u}.

In certain embodiments, R⁴ is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, R⁴ is C₁₋₆ aminoalkyl (e.g., C₁₋₆ alkyl groups substituted with one or more amino groups), wherein the aminoalkyl is optionally substituted with one or more R^{u}.

In certain embodiments, R⁴ is C₆₋₁₄ aryl (e.g., phenyl or naphthyl), wherein the aryl is optionally substituted with one or more R^{u}.

In certain embodiments, R⁴ is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, R⁴ is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, R⁴ is or 3- to 10-membered heterocyclyl (e.g., tetrahydrofuranyl, piperidinyl, or tetrahydropyranyl), wherein the heterocyclyl is optionally substituted with one or more R^{u}.

In certain embodiments, R⁶ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, R⁶ is C₁₋₆ alkyl (e.g., methyl, ethyl, or propyl), wherein the alkyl is optionally substituted with one or more R^{u}.

In certain embodiments, R⁶ is C₂₋₆ alkenyl (e.g., ethynyl, propenyl, butenyl, pentenyl, or hexenyl), wherein the alkenyl is optionally substituted with one or more R^{u}.

In certain embodiments, R⁶ is C₂₋₆ alkynyl (e.g., ethynyl, propynyl, butynyl, pentynyl, or hexynyl), wherein the alkynyl is optionally substituted with one or more R^{u}.

In certain embodiments, R⁶ is C₁₋₆haloalkyl (e.g., C₁₋₆ alkyl groups substituted with one or more halogen atom), wherein the haloalkyl is optionally substituted with one or more R^{u}.

In certain embodiments, R⁶ is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, R⁶ is C₁₋₆ aminoalkyl (e.g., C₁₋₆ alkyl groups substituted with one or more amino groups), wherein the aminoalkyl is optionally substituted with one or more R^{u}.

In certain embodiments, R⁶ is C₆₋₁₄ aryl (e.g., phenyl or naphthyl), wherein the aryl is optionally substituted with one or more R^{u}.

In certain embodiments, R⁶ is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, R⁶ is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, R⁶ is or 3- to 10-membered heterocyclyl (e.g., tetrahydrofuranyl, piperidinyl, or tetrahydropyranyl), wherein the heterocyclyl is optionally substituted with one or more R^{u}.

In certain embodiments, each R⁵ independently is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, each R⁵ independently is halogen (e.g., F, Cl, or Br).

In certain embodiments, each R⁵ independently is C₁₋₆ alkyl (e.g., methyl, ethyl, or propyl), wherein the alkyl is optionally substituted with one or more R^{u}.

In certain embodiments, each R⁵ independently is C₂₋₆ alkenyl (e.g., ethynyl, propenyl, butenyl, pentenyl, or hexenyl), wherein the alkenyl is optionally substituted with one or more R^{u}.

In certain embodiments, each R⁵ independently is C₂₋₆ alkynyl (e.g., ethynyl, propynyl, butynyl, pentynyl, or hexynyl), wherein the alkynyl is optionally substituted with one or more R^{u}.

In certain embodiments, each R⁵ is independently C₁₋₆haloalkyl (e.g., C₁₋₆ alkyl groups substituted with one or more halogen atom), wherein the haloalkyl is optionally substituted with one or more R^{u}.

In certain embodiments, each R⁵ independently is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, each R⁵ independently is C₁₋₆ aminoalkyl (e.g., C₁₋₆ alkyl groups substituted with one or more amino groups), wherein the aminoalkyl is optionally substituted with one or more R^{u}.

In certain embodiments, each R⁵ independently is C₆₋₁₄ aryl (e.g., phenyl or naphthyl), wherein the aryl is optionally substituted with one or more R^{u}.

In certain embodiments, each R⁵ independently is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, each R⁵ independently is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, each R⁵ independently is 3- to 10-membered heterocyclyl (e.g., tetrahydrofuranyl, piperidinyl, or tetrahydropyranyl), wherein the heterocyclyl is optionally substituted with one or more R^{u}.

In certain embodiments, R³ is —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d} (e.g., R^{a} is methyl or ethyl, R^{b} is H, R^{c} is H, and R^{d} is H).

In certain embodiments, X is selected from —C(R⁷)₂—, —O—, —NR⁸—, and —S— (e.g., R⁷ is H, halogen, or C₁₋₆ alkyl; and R⁸ is H or C₁₋₆ alkyl).

In certain embodiments, X is selected from —CH₂—, —CH(CH₃)—, —CHCl—, —CHBr—, —NH—, or —NCH₃—.

In certain embodiments, X is O.

In certain embodiments, R⁷ is hydrogen, halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, R⁷ is halogen (e.g., F, Cl, or Br).

In certain embodiments, R⁷ is C₁₋₆ alkyl (e.g., methyl, ethyl, or propyl), wherein the alkyl is optionally substituted with one or more R^{u}.

In certain embodiments, R⁷ is C₂₋₆ alkenyl (e.g., ethynyl, propenyl, butenyl, pentenyl, or hexenyl), wherein the alkenyl is optionally substituted with one or more R^{u}.

In certain embodiments, R⁷ is C₂₋₆ alkynyl (e.g., ethynyl, propynyl, butynyl, pentynyl, or hexynyl), wherein the alkynyl is optionally substituted with one or more R^{u}.

In certain embodiments, R⁷ is C₁₋₆haloalkyl (e.g., C₁₋₆ alkyl groups substituted with one or more halogen atom), wherein the haloalkyl is optionally substituted with one or more R^{u}.

In certain embodiments, R⁷ is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, R⁷ is C₁₋₆ aminoalkyl (e.g., C₁₋₆ alkyl groups substituted with one or more amino groups), wherein the aminoalkyl is optionally substituted with one or more R^{u}.

In certain embodiments, R⁷ is C₆₋₁₄ aryl (e.g., phenyl or naphthyl), wherein the aryl is optionally substituted with one or more R^{u}.

In certain embodiments, R⁷ is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, R⁷ is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, R⁷ is or 3- to 10-membered heterocyclyl (e.g., tetrahydrofuranyl, piperidinyl, or tetrahydropyranyl), wherein the heterocyclyl is optionally substituted with one or more R^{u}.

In certain embodiments, R⁷ is —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d} (e.g., R^{a} is methyl or ethyl, R^{b} is H, R^{c} is H, and R^{d} is H).

In certain embodiments, R⁸ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more R^{u} (e.g., R^{u} is F, Cl, Br, I, —CN, —NO₂, methyl, or ethyl).

In certain embodiments, R⁸ is C₁₋₆ alkyl (e.g., methyl, ethyl, or propyl), wherein the alkyl is optionally substituted with one or more R^{u}.

In certain embodiments, R⁸ is C₂₋₆ alkenyl (e.g., ethynyl, propenyl, butenyl, pentenyl, or hexenyl), wherein the alkenyl is optionally substituted with one or more R^{u}.

In certain embodiments, R⁸ is C₂₋₆ alkynyl (e.g., ethynyl, propynyl, butynyl, pentynyl, or hexynyl), wherein the alkynyl is optionally substituted with one or more R^{u}.

In certain embodiments, R⁸ is C₁₋₆haloalkyl (e.g., C₁₋₆ alkyl groups substituted with one or more halogen atom), wherein the haloalkyl is optionally substituted with one or more R^{u}.

In certain embodiments, R⁸ is C₁₋₆ hydroxyalkyl (e.g., C₁₋₆ alkyl groups substituted with one or more hydroxyl groups), wherein the hydroxyalkyl is optionally substituted with one or more R^{u}.

In certain embodiments, R⁸ is C₁₋₆ aminoalkyl (e.g., C₁₋₆ alkyl groups substituted with one or more amino groups), wherein the aminoalkyl is optionally substituted with one or more R^{u}.

In certain embodiments, R⁸ is C₆₋₁₄ aryl (e.g., phenyl or naphthyl), wherein the aryl is optionally substituted with one or more R^{u}.

In certain embodiments, R⁸ is 5- to 14-membered heteroaryl (e.g., pyrrolyl, furanyl, thiophenyl, pyridinyl, pyridazinyl, pyrimidinyl, or pyrazinyl), wherein the heteroaryl is optionally substituted with one or more R^{u}.

In certain embodiments, R⁸ is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl), wherein the carbocyclyl is optionally substituted with one or more R^{u}.

In certain embodiments, R⁸ is or 3- to 10-membered heterocyclyl (e.g., tetrahydrofuranyl, piperidinyl, or tetrahydropyranyl), wherein the heterocyclyl is optionally substituted with one or more R^{u}.

In certain embodiments, each R^{u} independently is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In certain embodiments, each R^{u} independently is halogen (e.g., F, Cl, or Br).

In certain embodiments, each R^{u} independently is C₁₋₆ alkyl (e.g., methyl, ethyl, or propyl) optionally submituted with one or more the aforementioned substitutents.

In certain embodiments, each R^{u} independently is C₂₋₆ alkenyl (e.g., ethynyl, propenyl, butenyl, pentenyl, or hexenyl) optionally submituted with one or more the aforementioned substitutents.

In certain embodiments, each R^{u} independently is C₂₋₆ alkynyl (e.g., ethynyl, propynyl, butynyl, pentynyl, or hexynyl) optionally submituted with one or more the aforementioned substitutents.

In certain embodiments, each R^{u} independently is C₁₋₆ haloalkyl (e.g., C₁₋₆ alkyl groups substituted with one or more halogen atom) optionally submituted with one or more the aforementioned substitutents.

In certain embodiments, each R^{u} independently is C₁₋₆ hydroxyalkyl (e.g., C₁₋₆ alkyl groups substituted with one or more hydroxyl groups) optionally submituted with one or more the aforementioned substitutents.

In certain embodiments, each R^{u} independently is C₁₋₆ aminoalkyl (e.g., C₁₋₆ alkyl groups substituted with one or more amino groups) optionally submituted with one or more the aforementioned substitutents.

In certain embodiments, each R^{u} independently is C₆₋₁₄ aryl (e.g., phenyl or naphthyl) optionally submituted with one or more the aforementioned substitutents.

In certain embodiments, each R^{u} independently is 5- to 14-membered heteroaryl (e.g., pyrrolyl, furanyl, thiophenyl, pyridinyl, pyridazinyl, pyrimidinyl, or pyrazinyl) optionally submituted with one or more the aforementioned substitutents.

In certain embodiments, each R^{u} independently is C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl) optionally submituted with one or more the aforementioned substitutents.

In certain embodiments, each R^{u} independently is 3- to 10-membered heterocyclyl (e.g., tetrahydrofuranyl, piperidinyl, or tetrahydropyranyl) optionally submituted with one or more the aforementioned substitutents.

In certain embodiments, each R^{u} independently is —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂R, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d} (e.g., R^{a} is methyl or ethyl, R^{b} is H, R^{c} is H, and R^{d} is H).

In certain embodiments, two R^{u} are taken together to form an oxo; or two R^{u}, together with the one or more intervening atoms, form a C₆₋₁₄ aryl, 5- to 14-membered heteroaryl, C₃₋₁₀ carbocyclyl, or 3- to 10-membered heterocyclyl.

In certain embodiments, two R^{u}, together with the one or more intervening atoms (e.g., C) form a C₆₋₁₄ aryl (e.g., phenyl or naphthyl).

In certain embodiments, two R^{u}, together with the one or more intervening atoms (e.g., N, O, or S) form a 5- to 14-membered heteroaryl (e.g., pyrrolyl, furanyl, thiophenyl, pyridinyl, pyridazinyl, pyrimidinyl, or pyrazinyl).

In certain embodiments, two R^{u}, together with the one or more intervening atoms (e.g., C) form a C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl).

In certain embodiments, two R^{u}, together with the one or more intervening atoms (e.g., N, O, or S) form a 3- to 10-membered heterocyclyl (e.g., tetrahydrofuranyl, piperidinyl, or tetrahydropyranyl).

In certain embodiments, each R^{a} is independently C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In certain embodiments, each R^{a} is independently C₁₋₆ alkyl (e.g., methyl, ethyl, or propyl) optionally submituted with one or more the aforementioned substitutents.

In certain embodiments, each R^{a} is independently C₂₋₆ alkenyl (e.g., ethynyl, propenyl, butenyl, pentenyl, or hexenyl) optionally submituted with one or more the aforementioned substitutents.

In certain embodiments, each R^{a} is independently C₂₋₆ alkynyl (e.g., ethynyl, propynyl, butynyl, pentynyl, or hexynyl) optionally submituted with one or more the aforementioned substitutents.

In certain embodiments, each R^{a} is independently C₁₋₆haloalkyl (e.g., C₁₋₆ alkyl groups substituted with one or more halogen atom) optionally submituted with one or more the aforementioned substitutents.

In certain embodiments, each R^{a} is independently C₁₋₆ hydroxyalkyl (e.g., C₁₋₆ alkyl groups substituted with one or more hydroxyl groups) optionally submituted with one or more the aforementioned substitutents.

In certain embodiments, each R^{a} is independently C₁₋₆ aminoalkyl (e.g., C₁₋₆ alkyl groups substituted with one or more amino groups) optionally submituted with one or more the aforementioned substitutents.

In certain embodiments, each R^{a} is independently C₆₋₁₄ aryl (e.g., phenyl or naphthyl).

In certain embodiments, each R^{a} is independently 5- to 14-membered heteroaryl (e.g., pyrrolyl, furanyl, thiophenyl, pyridinyl, pyridazinyl, pyrimidinyl, or pyrazinyl) optionally submituted with one or more the aforementioned substitutents.

In certain embodiments, each R^{a} is independently C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl) optionally submituted with one or more the aforementioned substitutents.

In certain embodiments, each R^{a} is independently or 3- to 10-membered heterocyclyl (e.g., tetrahydrofuranyl, piperidinyl, or tetrahydropyranyl) optionally submituted with one or more the aforementioned substitutents.

In certain embodiments, each R^{b} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In certain embodiments, each R^{b} is independently hydrogen.

In certain embodiments, each R^{b} is independently C₁₋₆ alkyl (e.g., methyl, ethyl, or propyl) optionally submituted with one or more the aforementioned substitutents.

In certain embodiments, each R^{b} is independently C₂₋₆ alkenyl (e.g., ethynyl, propenyl, butenyl, pentenyl, or hexenyl) optionally submituted with one or more the aforementioned substitutents.

In certain embodiments, each R^{b} is independently C₂₋₆ alkynyl (e.g., ethynyl, propynyl, butynyl, pentynyl, or hexynyl) optionally submituted with one or more the aforementioned substitutents.

In certain embodiments, each R^{b} is independently C₁₋₆haloalkyl (e.g., C₁₋₆ alkyl groups substituted with one or more halogen atom) optionally submituted with one or more the aforementioned substitutents.

In certain embodiments, each R^{b} is independently C₁₋₆ hydroxyalkyl (e.g., C₁₋₆ alkyl groups substituted with one or more hydroxyl groups) optionally submituted with one or more the aforementioned substitutents.

In certain embodiments, each R^{b} is independently C₁₋₆ aminoalkyl (e.g., C₁₋₆ alkyl groups substituted with one or more amino groups) optionally submituted with one or more the aforementioned substitutents.

In certain embodiments, each R^{b} is independently C₆₋₁₄ aryl (e.g., phenyl or naphthyl) optionally submituted with one or more the aforementioned substitutents.

In certain embodiments, each R^{b} is independently 5- to 14-membered heteroaryl (e.g., pyrrolyl, furanyl, thiophenyl, pyridinyl, pyridazinyl, pyrimidinyl, or pyrazinyl) optionally submituted with one or more the aforementioned substitutents.

In certain embodiments, each R^{b} is independently C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl) optionally submituted with one or more the aforementioned substitutents.

In certain embodiments, each R^{b} is independently or 3- to 10-membered heterocyclyl (e.g., tetrahydrofuranyl, piperidinyl, or tetrahydropyranyl) optionally submituted with one or more the aforementioned substitutents.

In certain embodiments, each R^{c} and R^{d} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

In certain embodiments, each R^{c} and R^{d} is independently C₁₋₆ alkyl (e.g., methyl, ethyl, or propyl) optionally submituted with one or more the aforementioned substitutents.

In certain embodiments, each R^{c} and R^{d} is independently C₂₋₆ alkenyl (e.g., ethynyl, propenyl, butenyl, pentenyl, or hexenyl) optionally submituted with one or more the aforementioned substitutents.

In certain embodiments, each R^{c} and R^{d} is independently C₂₋₆ alkynyl (e.g., ethynyl, propynyl, butynyl, pentynyl, or hexynyl) optionally submituted with one or more the aforementioned substitutents optionally submituted with one or more the aforementioned substitutents.

In certain embodiments, each R and R^{d} is independently C₁₋₆haloalkyl (e.g., C₁₋₆ alkyl groups substituted with one or more halogen atom) optionally submituted with one or more the aforementioned substitutents.

In certain embodiments, each R^{c} and R^{d} is independently C₁₋₆ hydroxyalkyl (e.g., C₁₋₆ alkyl groups substituted with one or more hydroxyl groups) optionally submituted with one or more the aforementioned substitutents.

In certain embodiments, each R^{c} and R^{d} is independently C₁₋₆ aminoalkyl (e.g., C₁₋₆ alkyl groups substituted with one or more amino groups) optionally submituted with one or more the aforementioned substitutents.

In certain embodiments, each R and R^{d} is independently C₆₋₁₄ aryl (e.g., phenyl or naphthyl) optionally submituted with one or more the aforementioned substitutents.

In certain embodiments, each R^{c} and R^{d} is independently 5- to 14-membered heteroaryl (e.g., pyrrolyl, furanyl, thiophenyl, pyridinyl, pyridazinyl, pyrimidinyl, or pyrazinyl) optionally submituted with one or more the aforementioned substitutents.

In certain embodiments, each R^{b} is independently C₃₋₁₀ carbocyclyl (e.g., cyclopropyl, cyclopropenyl, cyclobutyl, cyclobutenyl, cyclopentyl, cyclopentenyl, cyclohexyl, cyclohexenyl, cyclohexadienyl) optionally submituted with one or more the aforementioned substitutents.

In certain embodiments, each R^{c} and R^{d} is independently or 3- to 10-membered heterocyclyl (e.g., tetrahydrofuranyl, piperidinyl, or tetrahydropyranyl) optionally submituted with one or more the aforementioned substitutents.

In certain embodiments, R^{c}, R^{d}, together with the nitrogen atom to which they are attached, form a 3- to 10-membered heterocyclyl optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl. In certain embodiments, R^{c}, R^{d}, together with the nitrogen atom to which they are attached form a 3- to 10-membered heterocyclyl (e.g., tetrahydrofuranyl, piperidinyl, or tetrahydropyranyl) optionally submituted with one or more the aforementioned substitutents.

In certain embodiments, the compound is of Formula I-a,

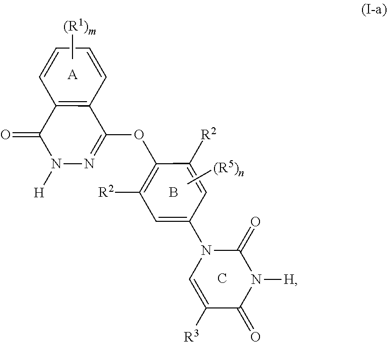

or pharmaceutically acceptable salts, solvates, or stereoisomers thereof.

In certain embodiments, the compound is selected from the compounds described in Table I, and pharmaceutically acceptable salts, stereoisomers, and solvates thereof.

In certain embodiments, the compound is selected from the compounds described in Table I.

TABLE I

| Com- | |
|---|---|
| puond | |
| Iden- | |
| tifier | Structure |
| a | 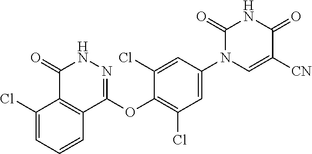 [SMILES: N#Cc1cn(-c2cc(Cl)c(Oc3n[nH]c(=O)c4c(Cl)cccc34)c(Cl)c2)c(=O)[nH]c1=O] |
| b | 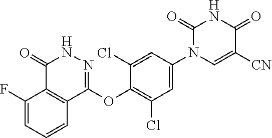 [SMILES: N#Cc1cn(-c2cc(Cl)c(Oc3n[nH]c(=O)c4c(F)cccc34)c(Cl)c2)c(=O)[nH]c1=O] |
| c | 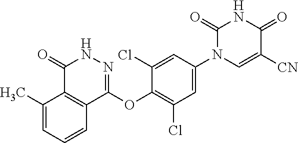 [SMILES: Cc1cccc2c(Oc3c(Cl)cc(-n4cc(C#N)c(=O)[nH]c4=O)cc3Cl)n[nH]c(=O)c12] |
| d | 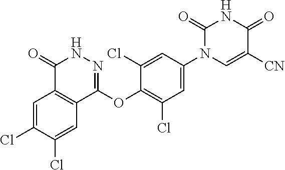 [SMILES: N#Cc1cn(-c2cc(Cl)c(Oc3n[nH]c(=O)c4cc(Cl)c(Cl)cc34)c(Cl)c2)c(=O)[nH]c1=O] |
| e | 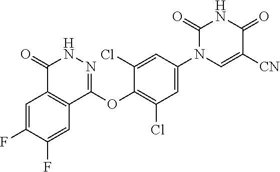 [SMILES: N#Cc1cn(-c2cc(Cl)c(Oc3n[nH]c(=O)c4cc(F)c(F)cc34)c(Cl)c2)c(=O)[nH]c1=O] |
| f | 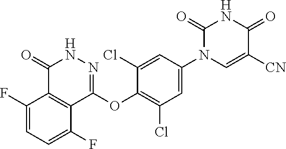 [SMILES: N#Cc1cn(-c2cc(Cl)c(Oc3n[nH]c(=O)c4c(F)ccc(F)c34)c(Cl)c2)c(=O)[nH]c1=O] |
| g | 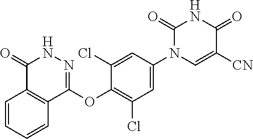 [SMILES: N#Cc1cn(-c2cc(Cl)c(Oc3n[nH]c(=O)c4ccccc34)c(Cl)c2)c(=O)[nH]c1=O] |
| h | 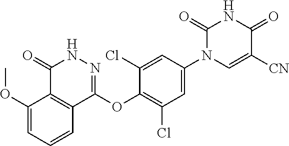 [SMILES: COc1cccc2c(Oc3c(Cl)cc(-n4cc(C#N)c(=O)[nH]c4=O)cc3Cl)n[nH]c(=O)c12] |
| i | 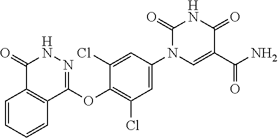 [SMILES: NC(=O)c1cn(-c2cc(Cl)c(Oc3n[nH]c(=O)c4ccccc34)c(Cl)c2)c(=O)[nH]c1=O] |
| j | 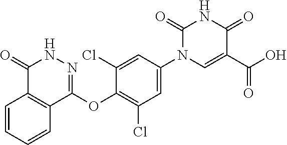 [SMILES: O=C(O)c1cn(-c2cc(Cl)c(Oc3n[nH]c(=O)c4ccccc34)c(Cl)c2)c(=O)[nH]c1=O] |

### Further Forms of Compounds Disclosed Herein

### Pharmaceutically Acceptable Salts

In some embodiments, the compounds disclosed herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In some embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In some embodiments, the compounds described herein exist as geometric isomers. In some embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In some embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

In some embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Thyroid receptor beta and thyroid receptor alpha coactivator recruitment assays were performed using LanthaScreen™ TR-FRET TR alpha or beta Coactivator Assay Kits (ThermoFIsher). The control agonist 3,3′,5-Triiodo-L-thyronine sodium salt, T3 (SIGMA) was used to validate the assay. Test compound and/or vehicle was incubated with the TR alpha LBD-GST or TR beta LBD-GST and coactivator peptide (Fluorescein-SRC2-2 (LKEKHKILHRLLQDSSSPV) (SEQ ID NO: 1)) for 30 minutes at RT. Determination of the amount of complex formed was read spectrofluorimetrically (excitation: 337 nm, emission: 520/490 nm) on a PHERAstar FS instrument (BMG). Test compound-induced increases in fluorescence greater than 50 percent relative to the T3 response were used to calculate EC50 values. Compounds were screened at 30, 10, 3, 1, 0.3, 0.1, and 0.03 μM.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

### Methods of Treatment

In one aspect, the present disclosure provides methods of activating thyroid hormone receptor (THR) β in a patient or a biological sample, the method comprising contacting the patient or biological sample with a compound described herein, or a pharmaceutically acceptable salt, stereoisomer, or solvate thereof.

In one aspect, the present disclosure provides methods of treating a liver disease or disorder, or a lipid disease or disorder, the method comprising administering to a patient in need thereof a compound described herein, or a pharmaceutically acceptable salt, stereoisomer, or solvate thereof.

In certain embodiments, the liver disease or disorder is nonalcoholic fatty liver disease (NAFLD), nonalcoholic steatohepatitis (NASH), or fatty liver disease.

In certain embodiments, the lipid disease or disorder is dyslipidemia, hyperlipidemia, hypertriglyceridemia, hypercholesterolemia, low high-density lipoprotein (HDL), or high low-density lipoprotein (LDL).

In certain embodiments, hypercholesterolemia is heterozygous familial hypercholesterolemia (HeFH) or homozygous familial hypercholesterolemia (HoFH).

In another aspect, the present disclosure provides uses of a compound described herein, or a pharmaceutically acceptable salt, stereoisomer, or solvate thereof, in the manufacture of a medicament for treating a liver disease or disorder, or a lipid disease or disorder.

In certain embodiments, the liver disease or disorder is NAFLD, NASH, or fatty liver disease.

In certain embodiments, the lipid disease or disorder is dyslipidemia, hyperlipidemia, hypertriglyceridemia, hypercholesterolemia, low HDL, or high LDL.

In certain embodiments, hypercholesterolemia is HeFH or HoFH.

In yet another aspect, the present disclosure provides compounds described herein, or a pharmaceutically acceptable salt, stereoisomer, or solvate thereof, or use in treating a liver disease or disorder, or a lipid disease or disorder.

In certain embodiments, the liver disease or disorder is NAFLD, NASH, or fatty liver disease.

In certain embodiments, the lipid disease or disorder is dyslipidemia, hyperlipidemia, hypertriglyceridemia, hypercholesterolemia, low HDL, or high LDL.

In certain embodiments, hypercholesterolemia is HeFH or HoFH.

### Synthesis of Compounds

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

In embodiments, a compound of described herein is prepared according to Scheme 1, Scheme 2, or Scheme 3 below:

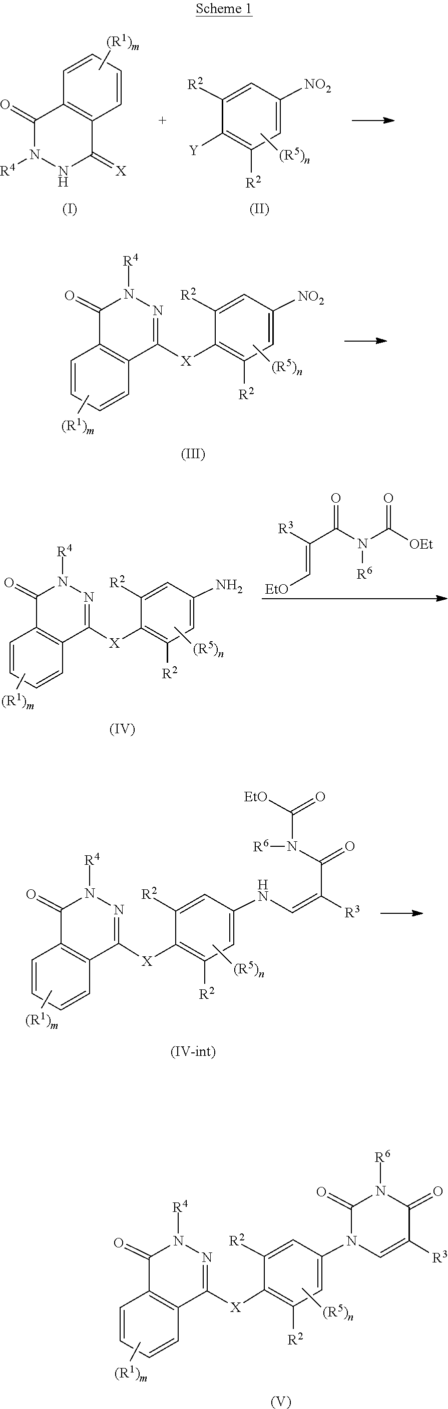

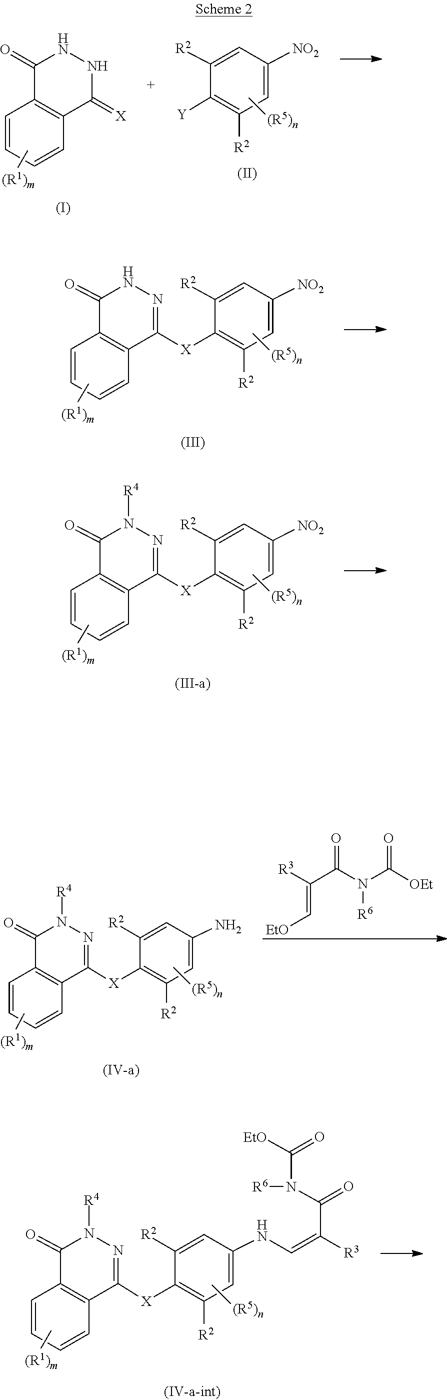

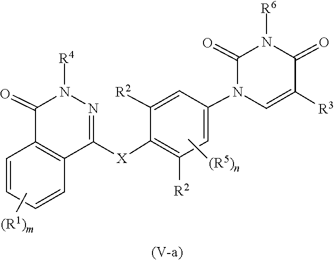

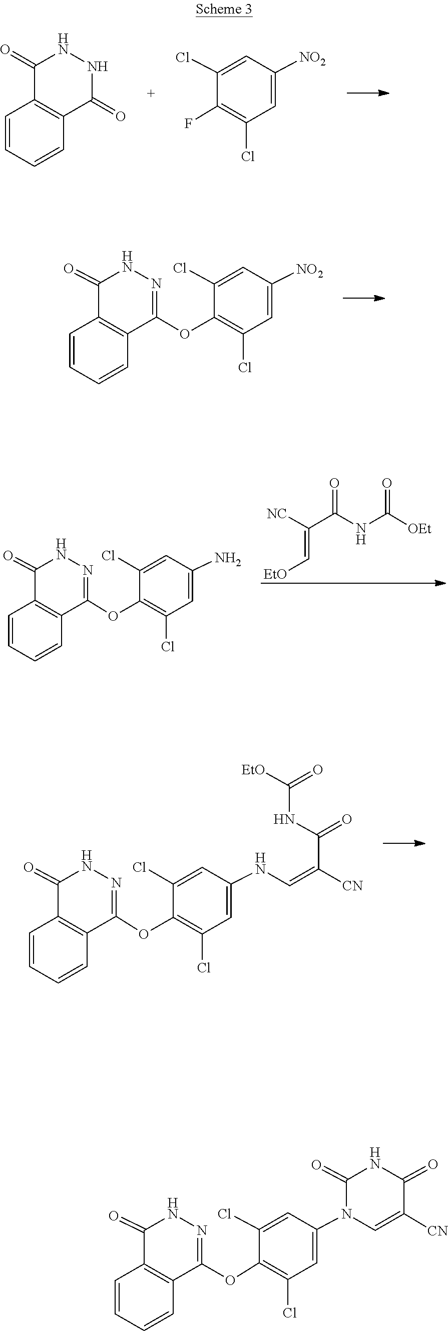

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

The resultant compounds of the present disclosure can be isolated and purified using techniques well known in the art.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

In some embodiments, the biological assay was performed with thyroid receptor beta and/or thyroid receptor alpha coactivator recruitment assays.

In some embodiments, 3,3′,5-Triiodo-L-thyronine sodium salt, T3 was used to as control agonist to validate the assay.

In some embodiments, the test compound and/or vehicle was incubated with the TR alpha LBD-GST or TR beta LBD-GST and coactivator peptide for 30 minutes at RT.

In some embodiments, the coactivator peptide is Fluorescein-SRC2-2 (LKEKHKILHRLLQDSSSPV) (SEQ ID NO: 1)).

In some embodiments, determination of the amount of complex formed was read spectrofluorimetrically (e.g., excitation: 337 nm, emission: 520/490 nm).

In some embodiments, the test compound-induced increases in fluorescence greater than 10, 20, 30, 40, 50, 60, 70, 80, or 90 percent relative to the T3 response.

In some embodiments, the test compound-induced increases in fluorescence greater than 50 percent relative to the T3 response were used to calculate EC₅₀ values.

In some embodiments, the compounds were screened at 30, 10, 3, 1, 0.3, 0.1, or 0.03 μM.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and/or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂-s alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and 1 or 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “alkylene,” “alkenylene,” “alkynylene,” “heteroalkylene,” “heteroalkenylene,” and “heteroalkynylene,” refer to a divalent radical of an alkyl, alkenyl, alkynyl group, heteroalkyl, heteroalkenyl, and heteroalkynyl group respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon divalent chain. “Alkylene,” “alkenylene,” “alkynylene,” “heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

“Aryl” also includes ring systems wherein the aryl ring, as defined above, is fused with one or more carbocyclyl or heterocyclyl groups wherein the radical or point of attachment is on the aryl ring, and in such instances, the number of carbon atoms continues to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁₋₆ alkyl, C₁₋₆ haloalkyl, cyano, hydroxy, C₁₋₆ alkoxy, and amino.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In some embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In some embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In some embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In some embodiments, the 5- to 6-membered heteroaryl has 1-3 ring heteroatoms independently selected from nitrogen, oxygen, and sulfur. In some embodiments, the 5- to 6-membered heteroaryl has 1-2 ring heteroatoms independently selected from nitrogen, oxygen, and sulfur. In some embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Carbocyclyl” refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or contains a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continues to designate the number of carbons in the carbocyclyl ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl include cyclopentyl (C₅) and cyclohexyl (C₅). Examples of C₃₋₆ cycloalkyl include the aforementioned C₅₋₆ cycloalkyl groups as well as cyclopropyl (C₃) and cyclobutyl (C₄). Examples of C₃₋₈ cycloalkyl include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3- to 10-membered heterocyclyl”). In heterocyclyl groups that contain one or more nitrogen atoms, the point of attachment can be a carbon or nitrogen atom, as valency permits.

A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, and in such instances, the number of ring members continues to designate the number of ring members in the heterocyclyl ring system. “Heterocyclyl” also includes ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In some embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In some embodiments, a heterocyclyl group is a 5- to 6-membered non-aromatic ring system having ring carbon atoms and 14 ring heteroatoms, wherein each heteroatom is independently selected from nitrogen, oxygen, and sulfur (“5- to 6-membered heterocyclyl”). In some embodiments, the 5- to 6-membered heterocyclyl has 1-3 ring heteroatoms selected from nitrogen, oxygen, and sulfur. In some embodiments, the 5- to 6-membered heterocyclyl has 1-2 ring heteroatoms selected from nitrogen, oxygen, and sulfur. In some embodiments, the 5- to 6-membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl.

Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, sulfur, boron, phosphorus, and silicon heteroatom, as valency permits. Hetero may be applied to any of the hydrocarbyl groups described above having from 1 to 5, and particularly from 1 to 3 heteroatoms.

“Acyl” refers to a radical —C(O)R, wherein R is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Representative acyl groups include, but are not limited to, formyl (—CHO), acetyl (—C(═O)CH₃), cyclohexylcarbonyl, cyclohexylmethylcarbonyl, benzoyl (—C(═O)Ph), and benzylcarbonyl (—C(═O)CH₂Ph).

“Acylamino” refers to a radical —NRC(═O)R, wherein each instance of R is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Exemplary “acylamino” groups include, but are not limited to, formylamino, acetylamino, cyclohexylcarbonylamino, cyclohexylmethyl-carbonylamino, benzoylamino and benzylcarbonylamino.

“Acyloxy” refers to a radical —OC(═O)R, wherein R is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Representative examples include, but are not limited to, formyl, acetyl, cyclohexylcarbonyl, cyclohexylmethylcarbonyl, benzoyl and benzylcarbonyl.

“Alkoxy” refers to the group —OR wherein R is substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, e.g., with between 1 and 6 carbon atoms. Further particular alkoxy groups have between 1 and 4 carbon atoms.

“Azido” refers to the radical —N₃.

“Carbamoyl” or “amido” refers to the radical —C(═O)NH₂.

“Amino” refers to the radical —NH₂.

“Oxo” refers to ═O.

“Thioketo” refers to the group ═S.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” or “(S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.10% by weight (R)-compound. In certain embodiments, the weights are based upon total weight of compound.

In the compositions provided herein, an enantiomerically pure compound or a pharmaceutically acceptable salt, solvate, or hydrate thereof can be present with other active or inactive ingredients. For example, a pharmaceutical composition comprising enantiomerically pure (R)-compound can comprise, for example, about 90% by weight excipient and about 10% by weight enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% by weight excipient and about 10% by weight enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset).

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is plus or minus certain percentages of the value stated, e.g., 5%, 10%, or 15% of the value stated. For example, about 100 would include 95 to 105, 90 to 110, or 85 to 115.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1—Synthetic Procedures

### 4-(2,6-dichloro-4-nitrophenoxy)phthalazin-1(2H)-one (Compound 7g)

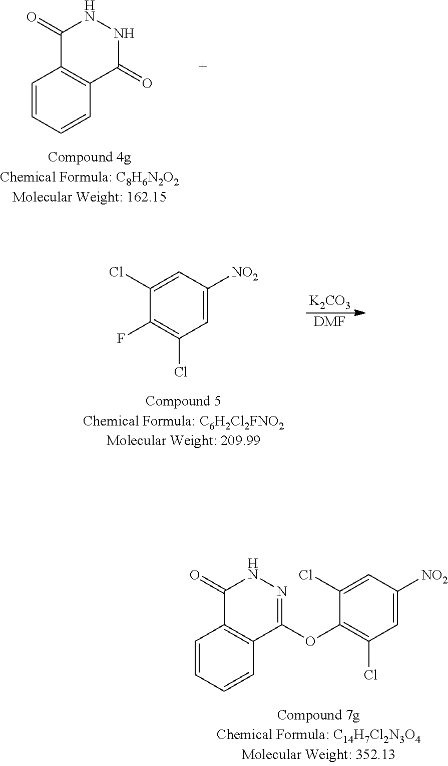

Potassium carbonate (2.60 g, 18.50 mmol) was added to the solution of phthalic hydrazide (Compound 4g, 2.00 g, 12.33 mmol) and Compound 5 (1.81 g, 8.63 mmol) in DMF (20 mL) and the mixture was stirred at room temperature for 3 hours before the reaction was quenched by water (60 mL). The resulting suspension was filtered. The filter cake was washed with water (20 mL) and dried under vacuum for 24 hours to afford crude compound 3 (2.98 g), which was further purified by slurry in ethyl acetate to afford Compound 7g as an off-white solid (2.20 g, yield: 50%).

Compound 7g: ¹H NMR (600 MHz, DMSO-d₆) δ 12.09 (s, 1H), 8.55 (s, 2H), 8.32 (d, J=7.5 Hz, 1H), 8.23 (d, J=7.8 Hz, 1H), 8.12-8.07 (m, 1H), 8.06-8.00 (m, 1H). ¹³C NMR (151 MHz, DMSO-d₆) δ 158.89, 150.39, 147.70, 145.43, 134.33, 133.40, 129.40, 129.09, 126.70, 124.63, 123.34, 122.77. LRMS for C₁₄H₈Cl₂N₃O₄ [M+H⁺] m/z=352.

### 4-(4-amino-2,6-dichlorophenoxy)phthalazin-1(2H)-one (INT Fg)

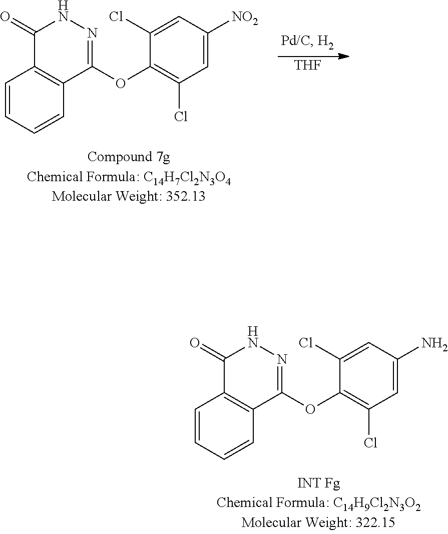 [SMILES: O=c1[nH]nc(Oc2c(Cl)cc([N+](=O)[O-])cc2Cl)c2ccccc12 ; Nc1cc(Cl)c(Oc2n[nH]c(=O)c3ccccc23)c(Cl)c1 ; "THF"]

Palladium on carbon (5%, 0.01 g) was added to the solution of Compound 7g (0.10 g, 0.28 mmol) in THF (10 mL). The solution was purged with nitrogen twice and then stirred under hydrogen atmosphere at room temperature for 5 hours. The mixture was filtered through a celite pad, and the filtrate was concentrated to dryness to afford INT Fg as a light brown solid (0.098 g, 107%).

ITN Fg: ¹H NMR (600 MHz, DMSO-d₆) δ 11.86 (s, 1H), 8.28 (d, J=7.7 Hz, 1H), 8.19 (d, J=7.8 Hz, 1H), 8.05-8.01 (m, 1H), 8.00-7.95 (m, 1H), 6.71 (s, 2H), 5.64 (s, 2H, NH₂). ¹³C NMR (151 MHz, DMSO-d₆) δ 158.88, 148.48, 148.03, 134.00, 133.90, 132.85, 128.88, 127.90, 126.46, 123.40, 123.37, 112.92. LRMS for C₁₄H₁₀Cl₂N₃O₂ [M+H⁺] m/z=322.

### (E,Z)-ethyl(2-cyano-3-((3,5-dichloro-4-((4-oxo-3,4-dihydrophthalazin-1-yl)oxy)phenyl)amino)acryloyl)carbamate (INT Gg)

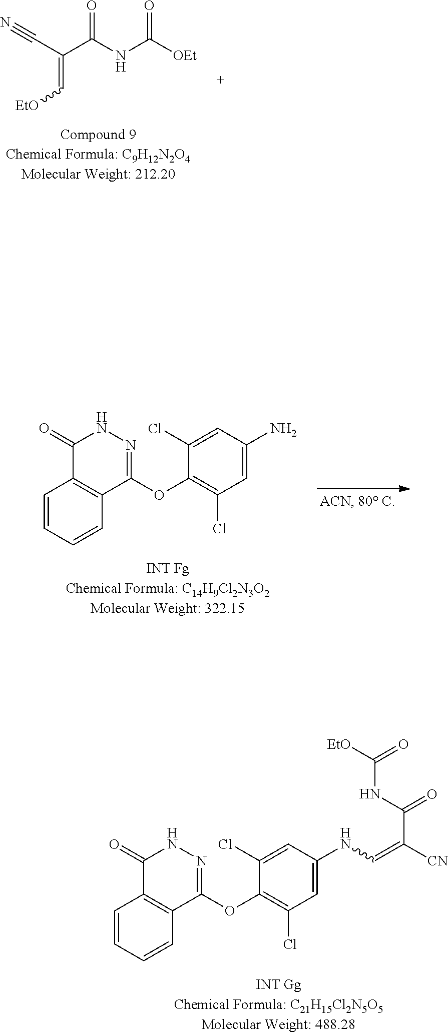 [SMILES: CCOC=C(C#N)C(=O)NC(=O)OCC ; Nc1cc(Cl)c(Oc2n[nH]c(=O)c3ccccc23)c(Cl)c1 ; CCOC(=O)NC(=O)C(C#N)=CNc1cc(Cl)c(Oc2n[nH]c(=O)c3ccccc23)c(Cl)c1]

Compound 9 (0.97 g, 4.57 mmol) was added to a solution of INT Fg (0.50 g, 20 1.55 mmol) in ACN (10 mL). The mixture was warmed to 80° C. for 5 hours. The mixture was cooled, and the resulting suspension was filtered. The filter cake was washed with ACN (10 mL) and dried under vacuum for 24 hours to afford a mixture of inseparable E,Z isomers of INT Gg as an off-white solid (0.52 g, yield: 69%).

INT Gg: ¹H NMR (600 MHz, DMSO-d₆, major isomer) δ 12.01 (s, 1H), 10.60 (d, J=13.5 Hz, 1H), 10.46 (s, 1H), 8.49 (d, J=13.5 Hz, 1H), 8.30 (d, J=7.9 Hz, 1H), 8.22 (d, J=7.8 Hz, 1H), 8.08 (t, J=7.0 Hz, 1H), 8.01 (t, J=7.1 Hz, 1H), 7.80 (s, 2H), 4.15 (q, J=7.1 Hz, 2H), 1.25 (t, J=7.1 Hz, 3H). ¹³C NMR (151 MHz, DMSO-d₆) δ 162.26, 158.91, 151.27, 151.20, 148.06, 141.21, 138.93, 134.21, 133.17, 128.98, 128.70, 128.70, 126.61, 123.39, 123.07, 118.31, 118.31, 115.58, 81.74, 60.95, 14.21. LRMS for C₂₁H₁₅C₂N₅O₅ [M+H⁺] m/z=488.

### 1-(3,5-dichloro-4-((4-oxo-3,4-dihydrophthalazin-1-yl)oxy)phenyl)-2,4-dioxo-1,2,3,4-tetrahydropyrimidine-5-carbonitrile (Compound g)

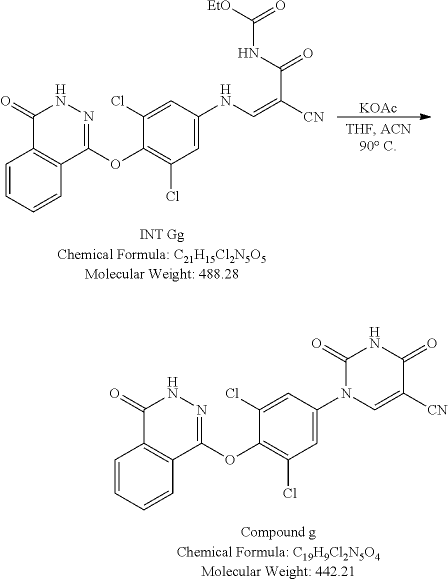 [SMILES: CCOC(=O)NC(=O)/C(C#N)=C\Nc1cc(Cl)c(Oc2n[nH]c(=O)c3ccccc23)c(Cl)c1 ; N#Cc1cn(-c2cc(Cl)c(Oc3n[nH]c(=O)c4ccccc34)c(Cl)c2)c(=O)[nH]c1=O ; "KOAc"]

INT Gg (0.20 g, 0.42 mmol) was added to a solution containing potassium acetate (0.09 g, 0.92 mmol), THF (4.0 mL), and ACN (2.0 mL). The mixture was purged with N₂, the flask was sealed, and heated to 90° C. for 6 days. The resulting suspension was cooled and filtered. The filter cake was purified by column chromatography (2→20% DCM:MeOH). The pure fractions were then concentrated to afford Compound g containing trace acetic acid as an off-white solid (0.040 g, yield: 22%).

Next, Compound g containing trace acetic acid (0.014 g, 0.032 mmol) was suspended in water (20 mL), heated to 80° C. for 30 min, cooled to room temperature, and stirred for 2 days. The suspension was filtered to provide purified Compound g as an off-white solid (0.0077 g, yield: 55%).

Compound g: ¹H NMR (600 MHz, DMSO-d₆) δ 12.31 (s, 1H), 12.05 (s, 1H), 8.92 (s, 1H), 8.32 (d, J=7.7 Hz, 1H), 8.25 (d, J=7.8 Hz, 1H), 8.09 (t, J=7.2 Hz, 1H), 8.03 (t, J=7.2 Hz, 1H), 7.92 (s, 2H). ¹³C NMR (151 MHz, DMSO-d₆) δ 158.84, 151.24, 147.96, 144.13, 134.26, 133.23, 129.04, 127.87, 127.87, 127.69, 126.66, 123.43, 123.02, 89.14. LRMS for C₂₁H₁₃Cl₂N₆O₄ [M+ACN+H⁺] m/z=483.

### Synthesis of 1-(3,5-dichloro-4-((5-chloro-4-oxo-3,4-dihydrophthalazin-1-yl)oxy)phenyl)-2,4-dioxo-1,2,3,4-tetrahydropyrimidine-5-carbonitrile (Compound a)

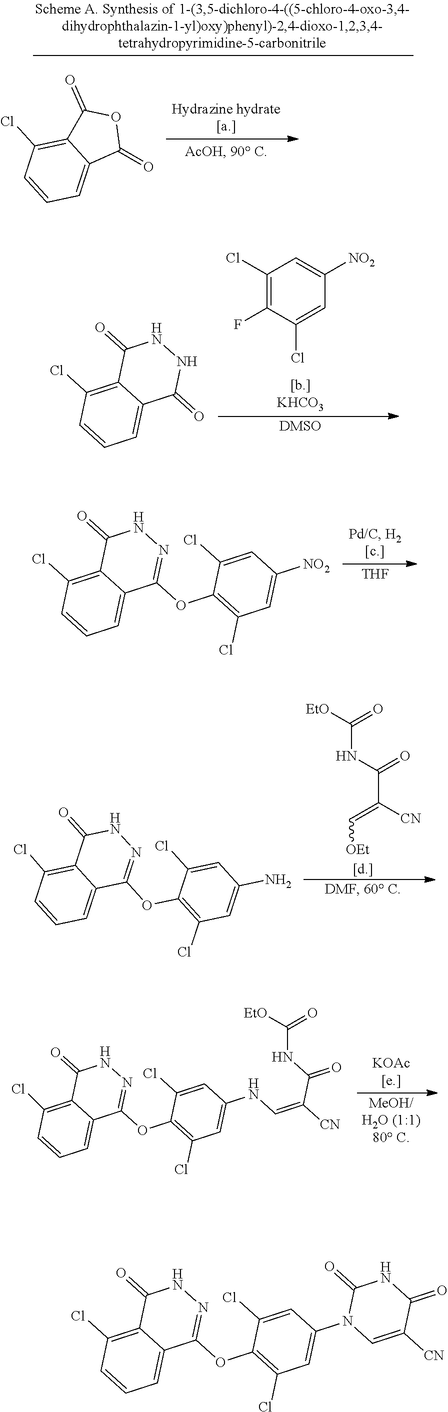

a. Synthesis of Hydrazine Reaction

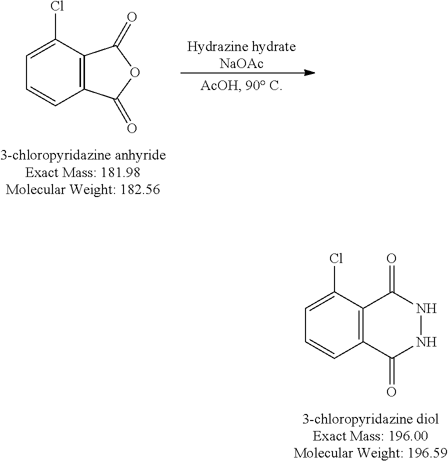 [SMILES: O=C1OC(=O)c2c(Cl)cccc21 ; "Hydrazine hydrateNaOAc" ; O=c1[nH][nH]c(=O)c2c(Cl)cccc12]

Sodium acetate (2.70 g, 33.0 mmol) was added to a solution of 3-chlorophthalic anhydride (5.02 g, 27.4 mmol) in acetic acid (25 mL) and stirred. Then hydrazine hydrate (1.56 mL, 33.0 mmol) was added dropwise and stirred for 15 mins. The mixture was warmed to 80-90° C. for 18 hours. The solid was filtered and washed with water. The solid was then subjected to aqueous KOH to dissolve the desired product and an impurity was filtered off as a solid. To the aqueous mother liquor, HCl was added dropwise until the pH ˜3.5, then the solid was filtered. This solid was isolated and dried under vacuum for 24 hours to afford Compound 4a as a white solid (4.53 g, yield: 84%).

Compound 4a: ¹H NMR (600 MHz, DMSO-d₆) δ 11.66 (s, 2H), 8.01 (d, J=8.0 Hz, 1H), 7.89 (d, J=6.5 Hz, 1H), 7.82 (dd, J=7.9 Hz, 1H). LRMS for C₈H₅ClN₂O₂ [M+H⁺] m/z=197).

b. Synthesis of SnAr Reaction

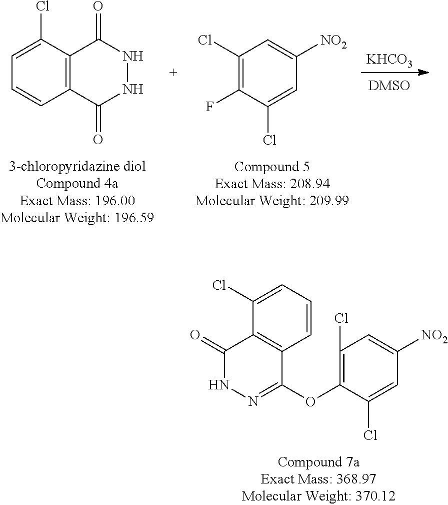

Potassium bicarbonate (0.928 g, 9.27 mmol) was added to the solution of 3-chlorophthalic hydrazide (Compound 4a, 1.49 g, 7.60 mmol) and Compound 5 (0.807 g, 3.84 mmol) in DMSO (100 mL) and the mixture was stirred at room temperature for 24 hours before the reaction was quenched by water 180 mL). The resulting suspension was filtered. The filter cake was washed with water (20 mL) and dried under vacuum for 24 hours to afford crude compound 7a which was further purified by slurry in ethyl acetate to afford Compound 7a as an off-white solid (1.04 g, yield: 74%).

Compound 7a: ¹H NMR (600 MHz, DMSO-d₆) δ 12.07 (s, 1H), 8.56 (s, 2H), 8.21 (dd, J=7.5, 1.5 Hz, 1H), 8.06-8.00 (m, 2H). LRMS for C₁₄H₆C₁₃N₃O₄ [M+H⁺] m/z=386)

c. Synthesis of Reduction Reaction

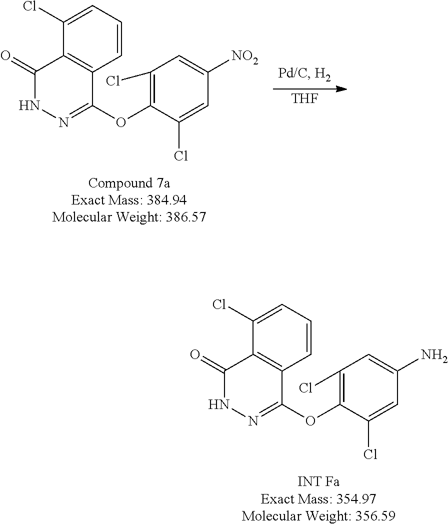 [SMILES: O=c1[nH]nc(Oc2c(Cl)cc([N+](=O)[O-])cc2Cl)c2cccc(Cl)c12 ; Nc1cc(Cl)c(Oc2n[nH]c(=O)c3c(Cl)cccc23)c(Cl)c1 ; "THF"]

Palladium on carbon (5%, 28.9 mg) was added to the solution of Compound 7a (0.201 g, 0.520 mmol) in THF (10 mL). The solution was purged with nitrogen three times and then stirred under hydrogen atmosphere at room temperature for 3.5 hours. The mixture was filtered through a celite pad, and the filtrate was concentrated to dryness to afford INT Fa as a light brown solid (0.071 g, 87.8%.) INT Fa: ¹H NMR (600 MHz, DMSO-d₆)¹H NMR (600 MHz, DMSO) δ 11.92 (s, 1H), 8.16 (dd, J=7.5, 1.5 Hz, 1H), 8.00-7.95 (m, 2H), 6.70 (s, 2H), 5.65 (s, 2H). LRMS for C₁₄H₈C₁₃N₃O₂ [M+H⁺] m/z=356.

d. Synthesis of Condensation Reaction

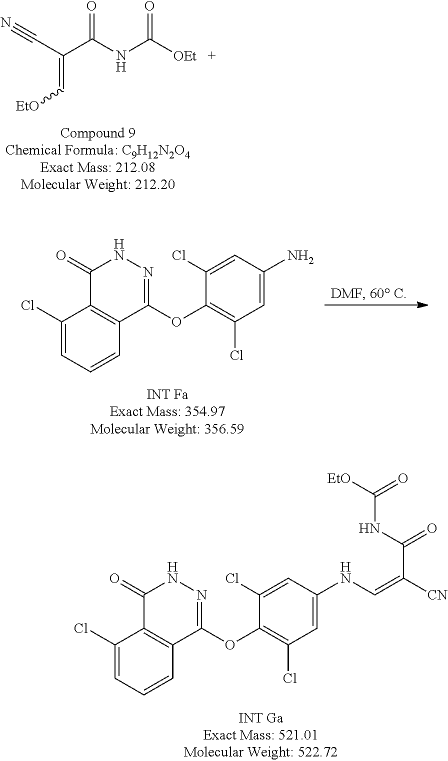

Compound 9 (56.4 mg, 0.26 mmol) was added to a solution of INT Fa (34.9 mg, 0.10 mmol) in DMF (1.0 mL). The mixture was warmed to 60° C. for 3.5 hours. The mixture was cooled, and water (1.0 mL) was added, the resulting suspension was filtered. The filter cake was washed with water (1.0 mL) and dried under vacuum for 24 hours to afford a mixture of inseparable E,Z isomers of INT Ga as an off-white solid (47.7 mg, yield: 86). INT Ga: ¹H NMR (600 MHz, DMSO-d₆) δ 12.00 (s, 1H), 10.61 (d, J=13.4 Hz, 1H), 10.47 (s, 1H), 8.48 (d, J=13.5 Hz, 1H), 8.20 (dd, J=7.6, 1.6 Hz, 1H), 8.04-7.98 (m, 2H), 7.79 (s, 2H), 4.15 (q, J=7.1 Hz, 2H), 1.25 (t, J=7.1 Hz, 3H). LRMS for C₂₁H₁₄C₁₃N₅O₅ [M+H⁺] m/z=522.

e. Synthesis of Cyclization Reaction

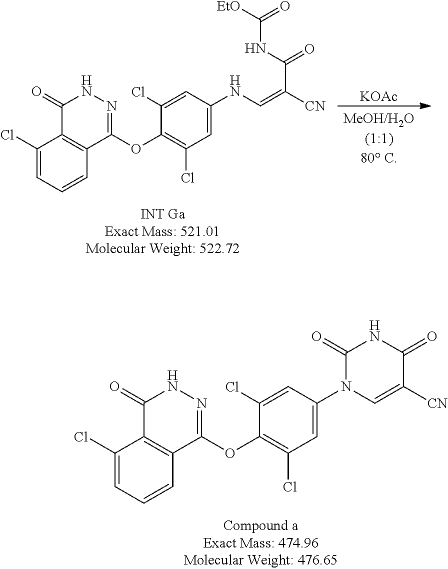 [SMILES: CCOC(=O)NC(=O)/C(C#N)=C\Nc1cc(Cl)c(Oc2n[nH]c(=O)c3c(Cl)cccc23)c(Cl)c1 ; N#Cc1cn(-c2cc(Cl)c(Oc3n[nH]c(=O)c4c(Cl)cccc34)c(Cl)c2)c(=O)[nH]c1=O ; "KOAc"]

INT Ga (39.3 mg, 0.08 mmol) was added to a solution containing potassium acetate (16.4 mg, 0.15 mmol), MeOH (1.0 mL), and water (1.0 mL). The mixture was purged with N₂, the flask was sealed, and heated to 80° C. for 6 hours. The resulting suspension was cooled and filtered. The filter cake was dried in the oven and resulted in an off-white solid (21.6 mg, yield: 87.8%).

Compound a: ¹H NMR (600 MHz, DMSO-d₆), δ 12.00 (s, 1H), 10.61 (d, J=13.4 Hz, 1H), 10.47 (s, 1H), 8.48 (d, J=13.5 Hz, 1H), 8.20 (dd, J=7.6, 1.6 Hz, 1H), 8.04-7.98 (m, 2H), 7.79 (s, 2H), 4.15 (q, J=7.1 Hz, 2H), 1.25 (t, J=7.1 Hz, 3H). LRMS for C₁₉H₈C₁₃N₅O₄ [M+H⁺] m/z=476.

### Synthesis of 1-(3,5-dichloro-4-((5-fluoro-4-oxo-3,4-dihydrophthalazin-1-yl)oxy) phenyl)-2,4-dioxo-1,2,3,4-tetrahydropyrimidine-5-carbonitrile (Compound b)

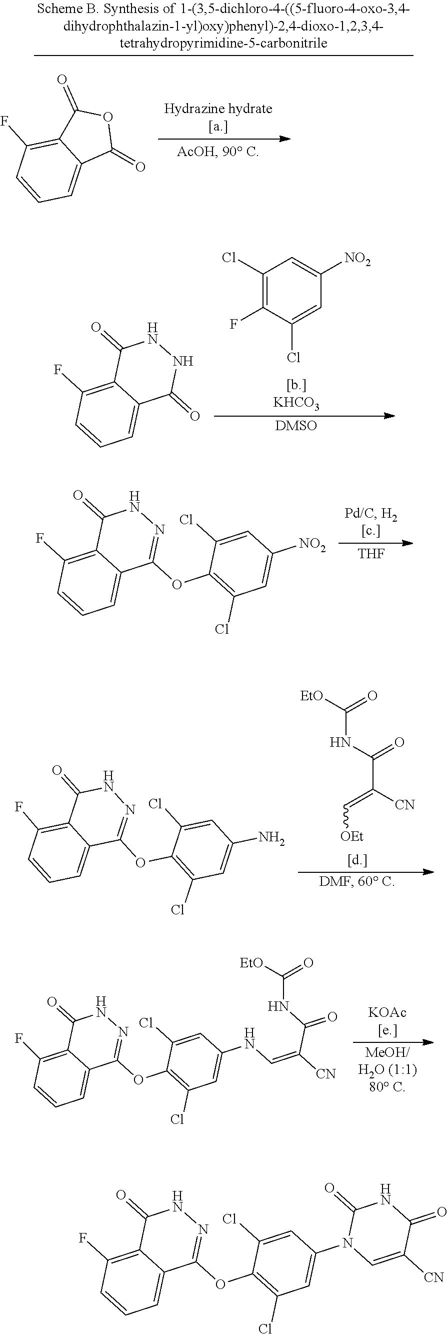

a. Synthesis of Hydrazine Reaction

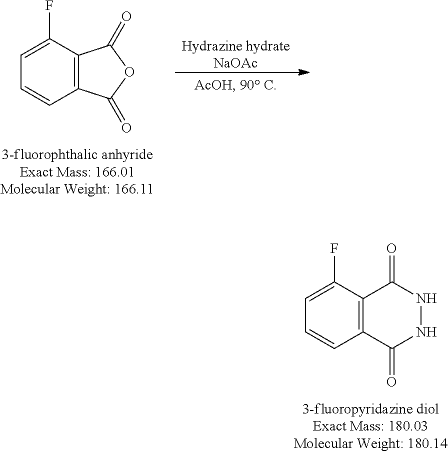 [SMILES: O=C1OC(=O)c2c(F)cccc21 ; "Hydrazine hydrateNaOAc" ; O=c1[nH][nH]c(=O)c2c(F)cccc12]

Sodium acetate (2.96 g, 36.0 mmol) was added to a solution of 3-fluorophthalic anhydride 5.07 g, 30.0 mmol) in acetic acid (25 mL) and stirred. Then hydrazine hydrate (1.75 mL, 36.0 mmol) was added dropwise and stirred for 15 mins. The mixture was warmed to 80-90° C. for 18 hours. The solid was filtered and washed with water and dried under vacuum for 24 hours to afford Compound 4b as a white solid (5.13 g, yield: 95%).

Compound 4b: ¹H NMR (600 MHz, DMSO-d₆) δ 11.57 (s, 2H), 7.89 (p, J=6.5 Hz, 2H), 7.69-7.62 (m, 1H). LRMS for C₈H₅FN₂O₂ [M+H⁺] m/z=181.

b. Synthesis of SnAr Reaction

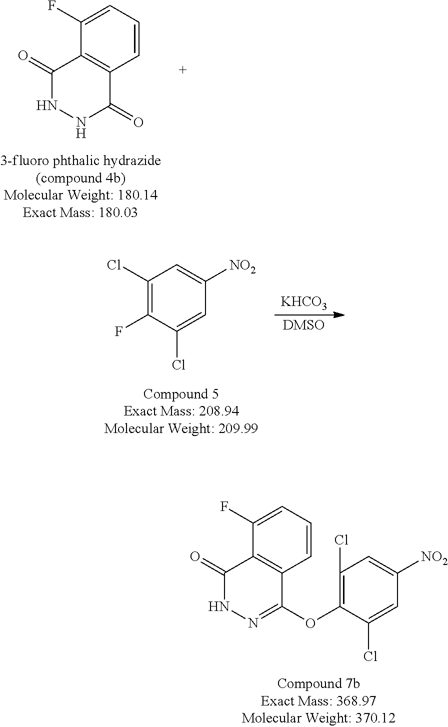

Potassium bicarbonate (2.01 g, 20.01 mmol) was added to the solution of 3-fluorophthalic hydrazide (Compound 4b, 3.08 g, 17.10 mmol) and Compound 5 (1.77 g, 8.44 mmol) in DMSO (300 mL) and the mixture was stirred at room temperature for 24 hours before the reaction was quenched by water (60 mL). The resulting suspension was filtered. The filter cake was washed with water (20 mL) and dried under vacuum for 24 hours to afford crude compound 7b (2.8242 g), which was further purified by slurry in ethyl acetate to afford Compound 7b as an off-white solid (501 g, yield: 16%).

Compound 7b: ¹H NMR (600 MHz, DMSO-d₆) δ 12.06 (s, 1H), 8.57 (d, J=4.0 Hz, 2H), 8.11 (td, J=8.0, 4.5 Hz, 1H), 8.06 (dd, J=8.0, 1.2 Hz, 1H), 7.82 (dd, J=11.3, 8.2 Hz, 1H). LRMS for C₁₄H₆Cl₂FN₃O₄ [M+H⁺] m/z=370.

c. Synthesis of Reduction Reaction

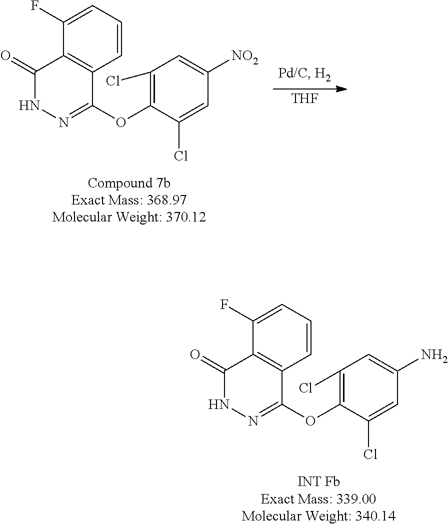 [SMILES: O=c1[nH]nc(Oc2c(Cl)cc([N+](=O)[O-])cc2Cl)c2cccc(F)c12 ; Nc1cc(Cl)c(Oc2n[nH]c(=O)c3c(F)cccc23)c(Cl)c1 ; "THF"]

Palladium on carbon (5%, 37 mg) was added to the solution of Compound 7b (0.301 g, 0.10 mmol) in THF (10 mL). The solution was purged with nitrogen twice and then stirred under hydrogen atmosphere at room temperature for 3 hours. The mixture was filtered through a celite pad, and the filtrate was concentrated to dryness to afford INT Fb as a light tan solid (0.283 g, 100%).

INT Fb: ¹H NMR (600 MHz, DMSO-d₆) δ 11.89 (s, 1H), 8.04 (td, J=8.0, 4.5 Hz, 1H), 8.00 (dd, J=8.0, 1.3 Hz, 1H), 7.78-7.73 (m, 1H), 6.70 (s, 2H), 5.65 (s, 2H). LRMS for C₁₄H₈Cl₂FN₃O₂ [M+H⁺] m/z=340.

d. Synthesis of Condensation Reaction

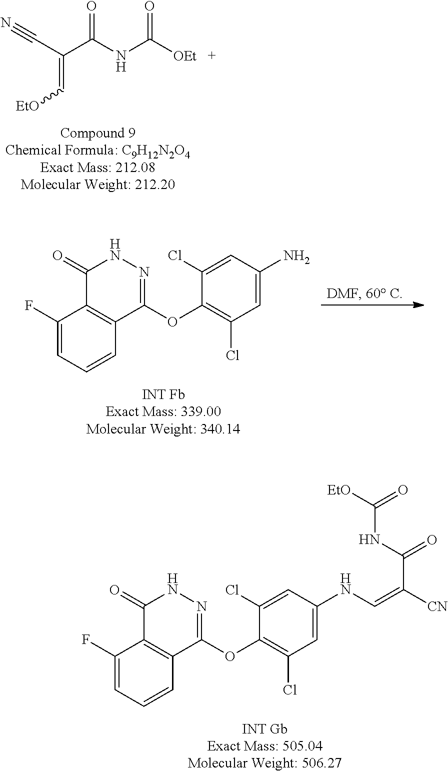

Compound 9 (91.8 mg, 0.43 mmol) was added to a solution of INT Fb (50.7 mg, 0.15 mmol) in DMF (1 mL). The mixture was warmed to 60° C. for 2 hours. The mixture was cooled, and water was added and the resulting suspension was filtered. The filter cake was washed with water (3 mL) and dried under vacuum for 24 hours to afford a mixture of inseparable E,Z isomers of INT Gb as an yellow solid (53.3 mg, yield: 76%).

INT Gb: ¹H NMR (600 MHz, DMSO-d₆) δ 11.97 (s, 1H), 10.61 (d, J=13.5 Hz, 1H), 10.47 (s, 1H), 8.48 (d, J=13.5 Hz, 1H), 8.08 (td, J=8.0, 4.5 Hz, 1H), 8.04 (d, J=6.7 Hz, 1H), 7.94 (d, J=12.6 Hz, 1H), 7.79 (s, 2H), 4.15 (q, J=7.1 Hz, 2H), 1.25 (t, J=7.1 Hz, 3H). LRMS for C₂₁H₁₄Cl₂FN₅O₅ [M+H⁺] m/z=506.

e. Synthesis of Cyclization Reaction

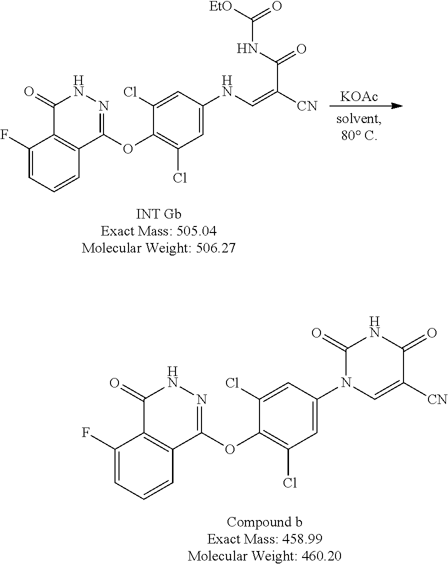 [SMILES: CCOC(=O)NC(=O)/C(C#N)=C\Nc1cc(Cl)c(Oc2n[nH]c(=O)c3c(F)cccc23)c(Cl)c1 ; N#Cc1cn(-c2cc(Cl)c(Oc3n[nH]c(=O)c4c(F)cccc34)c(Cl)c2)c(=O)[nH]c1=O ; "KOAc"]

INT Gb (38.5 mg, 0.078 mmol) was added to a solution containing potassium acetate (19.8 mg, 0.20 mmol), MeOH (1.0 mL), and water (1.0 mL). The mixture was purged with N₂, the flask was sealed, and heated to 60° C. for 2.5 hours. The resulting suspension was cooled, and water was added (2.0 mL) and filtered. The filter cake was washed with water then dried in a vacuum oven to afford Compound b as an off-white solid (12.1 mg, yield: 34%).

Compound b: ¹H NMR (600 MHz, DMSO-d₆) δ 12.32 (s, 1H), 12.01 (s, 1H), 8.92 (s, 1H), 8.10 (dt, J=8.0, 4.0 Hz, 1H), 8.06 (dd, J=8.0, 1.3 Hz, 1H), 7.92 (s, 2H), 7.80 (dd, J=10.0, 8.0 Hz, 1H). LRMS for C₁₉H₈Cl₂FN₅O₄ [M+H⁺] m/z=460.

### Synthesis of 1-(3,5-dichloro-4-((5-methyl-4-oxo-3,4-dihydrophthalazin-1-yl)oxy)phenyl)-2,4-dioxo-1,2,3,4-tetrahydropyrimidine-5-carbonitrile (Compound c)

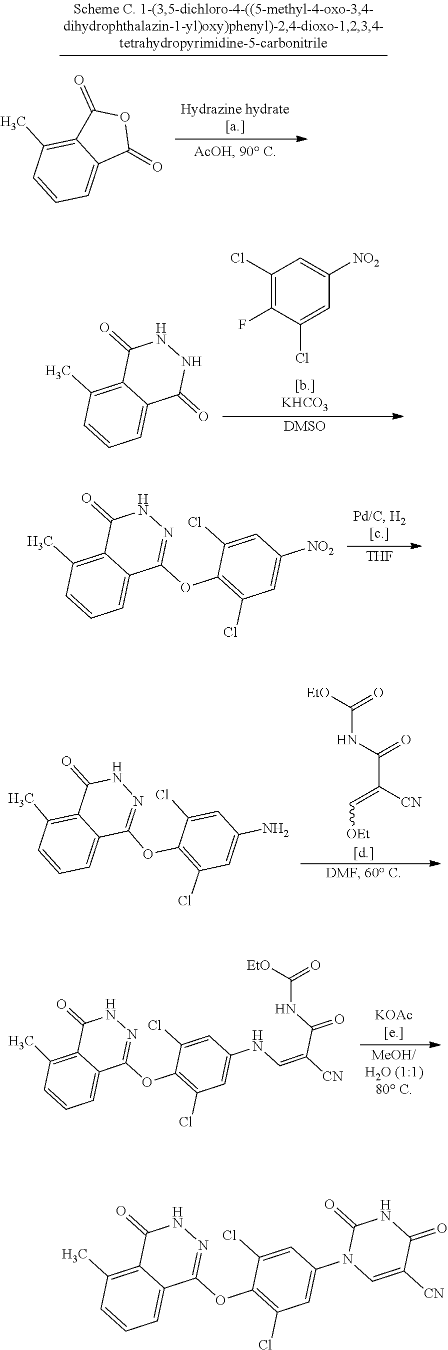

a. Synthesis of Hydrazine Reaction

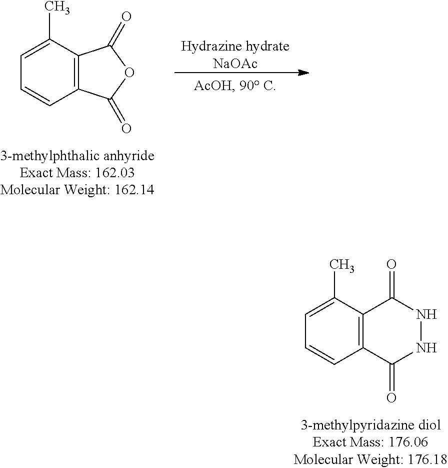

Sodium acetate (1.22 g, 14.9 mmol) was added to a solution of 3-methylphthalic anhydride (2.03 g, 12.5 mmol) in acetic acid (10 mL) and stirred. Then hydrazine hydrate (0.66 mL, 15.0 mmol) was added dropwise and stirred for 15 mins. The mixture was warmed to 80-90° C. for 18 hours. The solid was filtered and washed with water. Then water was added to the solid and added 5M KOH, until pH ˜11, the solid that remained was filtered off and discarded. The mother liquor was then acidified ˜2 and then the solid that formed was filtered. The solid filtered was then dried under vacuum for 24 hours to afford Compound 4c as a white solid (411 mg, yield: 96%).

Compound 4c: ¹H NMR (600 MHz, DMSO-d₆) δ 7.90 (d, J=7.3 Hz, 1H), 7.65 (t, J=7.7 Hz, 1H), 7.52 (d, J=6.3 Hz, 1H), 2.80 (s, 3H), 1.75 (s, 2H). LRMS for C₉H₈N₂O₂ [M+H⁺] m/z=177.

b. Synthesis of SnAr Reaction

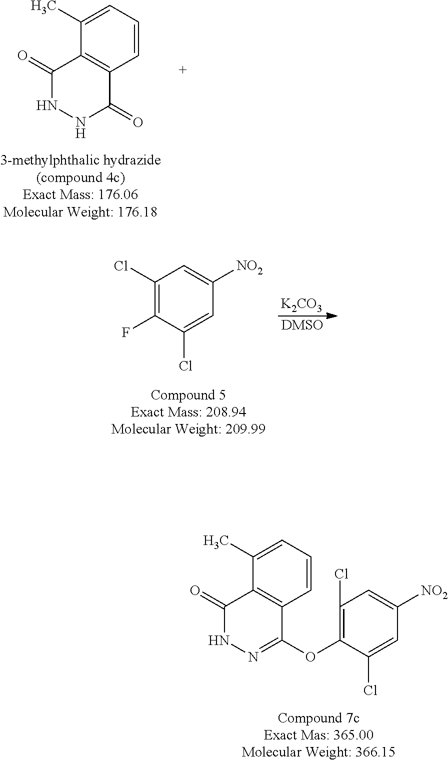

Potassium bicarbonate (263 mg, 2.63 mmol) was added to the solution of 3-methylphthalic hydrazide (Compound 4c, 350 mg, 1.79 mmol) and Compound 5 (226 mg, 1.08 mmol) in DMSO (150 mL) and the mixture was stirred at room temperature for 24 hours before the reaction was quenched by water (60 mL). The resulting suspension was filtered. The filter cake was washed with water (20 mL) and dried under vacuum for 24 hours to afford crude compound 7g, which was further purified by slurry in ethyl acetate to afford Compound 7c as an off-white solid (281 g, yield: 70%).

Compound 7c: ¹H NMR (600 MHz, DMSO-d₆) δ 11.86 (s, 1H), 8.55 (s, 2H), 8.07 (d, J=7.5 Hz, 1H), 7.93 (t, J=7.7 Hz, 1H), 7.78 (d, J=7.4 Hz, 1H), 2.86 (s, 3H). LRMS for C₁₅H₉Cl₂N₃O₄ [M+H⁺] m/z=366.

c. Synthesis of Reduction Reaction

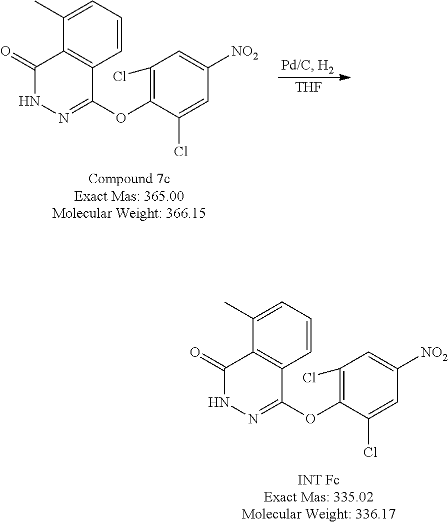 [SMILES: Cc1cccc2c(Oc3c(Cl)cc([N+](=O)[O-])cc3Cl)n[nH]c(=O)c12 ; "Pd/C, H2" ; Cc1cccc2c(Oc3c(Cl)cc([N+](=O)[O-])cc3Cl)n[nH]c(=O)c12 ; "THF"]

Palladium on carbon (5%, 44.8 mg) was added to the solution of Compound 7c (281.5 g, 0.77 mmol) in THF (10 mL). The solution was purged with nitrogen twice and then stirred under hydrogen atmosphere at room temperature for 24 hours. The mixture was filtered through a celite pad, and the filtrate was concentrated to dryness to afford INT Fe as a light brown solid (215 mg, 83%).

INT Fc: ¹H NMR (600 MHz, DMSO-d₆) δ 11.69 (s, 1H), 8.02 (d, J=6.7 Hz, 1H), 7.87 (t, J=7.8 Hz, 1H), 7.72 (d, J=7.5 Hz, 1H), 6.69 (s, 2H), 5.63 (s, 2H), 2.85 (s, 3H). LRMS for C₁₅H₁₁Cl₂N₃O₂ [M+H⁺] m/z=336.

d. Synthesis of Condensation Reaction

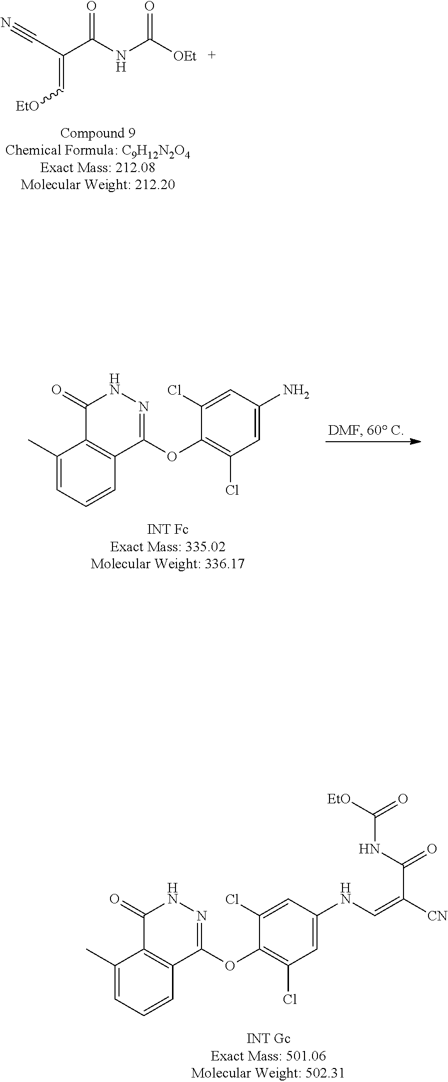

Compound 9 (65.4 mg, 0.31 mmol) was added to a solution of INT Fc (36.3 mg, 0.11 mmol) in DMF (2 mL). The mixture was warmed to 80° C. for 5 hours. The mixture was cooled, and the resulting suspension was filtered. The resulting suspension was cooled, and water was added (2.0 mL) and filtered. The filter cake was washed with water then dried in a vacuum oven to afford INT Gc as a pale yellow solid (36.2 mg, yield: 97%).

INT Gc: ¹H NMR (600 MHz, DMSO-d₆) δ 11.78 (s, 1H), 10.60 (d, J=13.5 Hz, 1H), 10.47 (s, 1H), 8.48 (d, J=13.5 Hz, 1H), 8.05 (d, J=7.8 Hz, 1H), 7.91 (d, J=7.9 Hz, 1H), 7.79 (s, 2H), 7.76 (d, J=7.4 Hz, 1H), 4.15 (q, J=7.1 Hz, 2H), 2.86 (s, 3H), 1.25 (t, J=7.1 Hz, 3H). LRMS for C₂₂H₁₇Cl₂N₅O₅ [M+H⁺] m/z=502.

e. Synthesis of Cyclization Reaction

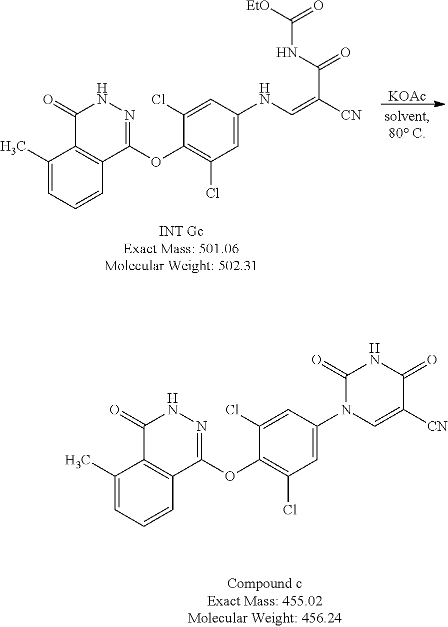 [SMILES: CCOC(=O)NC(=O)/C(C#N)=C\Nc1cc(Cl)c(Oc2n[nH]c(=O)c3c(C)cccc23)c(Cl)c1 ; Cc1cccc2c(Oc3c(Cl)cc(-n4cc(C#N)c(=O)[nH]c4=O)cc3Cl)n[nH]c(=O)c12 ; "KOAc"]

INT Gc (30.6 mg, 0.06 mmol) was added to a solution containing potassium acetate (13.6 mg, 0.14 mmol), MeOH (1.0 mL), and water (1.0 mL). The mixture was purged with N₂, the flask was sealed, and heated to 80° C. for 5 hours The resulting suspension was cooled, and water was added (2.0 mL) and filtered. The filter cake was washed with water then dried in a vacuum oven to afford Compound c as a pale yellow solid (12.0 mg, yield: 44%).

Compound c: ¹H NMR (600 MHz, DMSO-d₆) δ 12.31 (s, 1H), 11.82 (s, 1H), 8.92 (s, 1H), 8.07 (d, J=6.6 Hz, 1H), 7.93 (d, J=7.7 Hz, 1H), 7.91 (s, 2H), 7.77 (d, J=7.4 Hz, 1H), 2.86 (s, 3H). LRMS for C₂₀H₁₁Cl₂N₅O₄ [M+H⁺] m/z=456.

### Synthesis of 1-(3,5-dichloro-4-((6,7-dichloro-4-oxo-3,4-dihydrophthalazin-1-yl)oxy)phenyl)-2,4-dioxo-1,2,3,4-tetrahydropyrimidine-5-carbonitrile (Compound d)

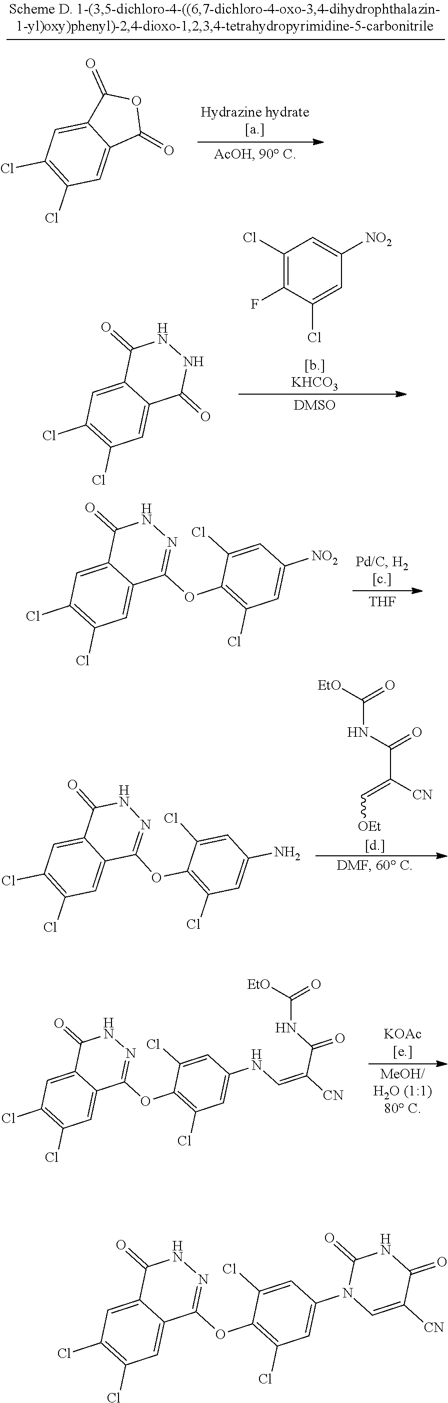

a. Synthesis of Hydrazine Reaction

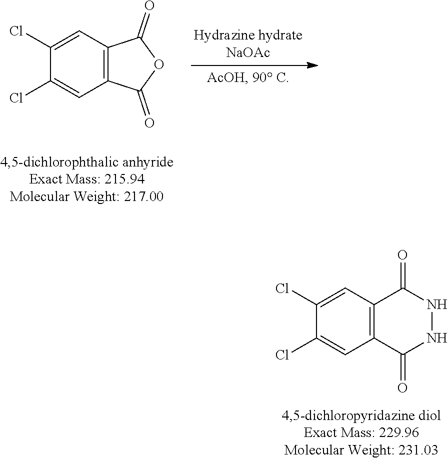 [SMILES: O=C1OC(=O)c2cc(Cl)c(Cl)cc21 ; O=c1[nH][nH]c(=O)c2cc(Cl)c(Cl)cc12]

Sodium acetate (1.36 g, 17.0 mmol) was added to a solution of 4,5-dichlorophthalic anhydride (3.01 g, 14.0 mmol) in acetic acid (16 mL) and stirred. Then hydrazine hydrate (0.81 mL, 17.0 mmol) was added dropwise and stirred for 15 mins. The mixture was warmed to 80-90° C. for 18 hours. The solid was filtered and washed with water. The solid was then subjected to aqueous KOH to dissolve the desired product and an impurity was filtered off as a solid. To the aqueous mother liquor, HCl was added dropwise until the pH ˜3.5, then the solid was filtered. This solid was isolated and dried under vacuum for 24 hours to afford Compound 4d as a white solid (3.31 g, yield: 96%).

Compound 4d: ¹H NMR (600 MHz, DMSO-d₆) δ 11.89 (s, 2H), 8.19 (s, 2H). LRMS for C₈H₄Cl₂N₂O₂ [M+H⁺] m/z=230.

b. Synthesis of SnAr Reaction

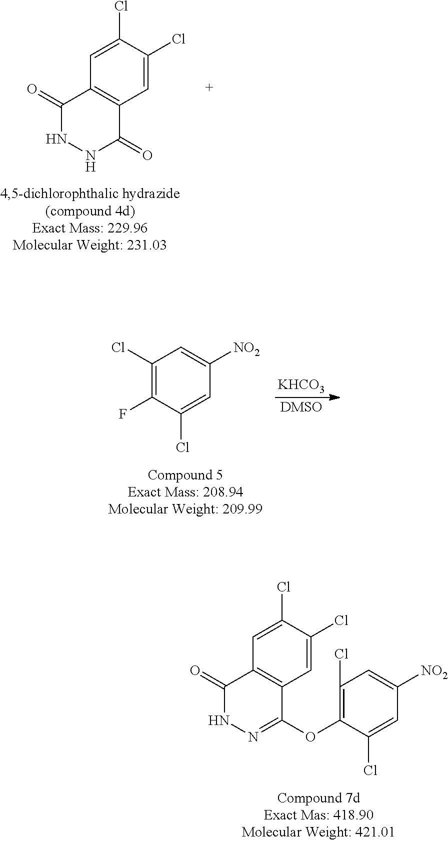

Potassium bicarbonate (421 mg, 4.1 mmol) was added to the solution of 4,5-dichlorophthalic hydrazide (Compound 4d, 290.1 mg, 1.26 mmol) and Compound 5 (375 mg, 1.79 mmol) in DMSO (100 mL) and the mixture was stirred at room temperature for 48 hours before the reaction was quenched by water (60 mL). The resulting suspension was filtered. The filter cake was washed with water (20 mL) and dried under vacuum for 24 hours to afford crude compound 7d, which was further purified by column chromatography (2 to 10% MeOH/DCM) Compound 7d as an off-white solid (310 mg, yield: 43%).

Compound 7d: ¹H NMR (600 MHz, DMSO-d₆) δ 12.33 (s, 1H), 8.58 (s, 2H), 8.51 (s, 1H), 8.45 (s, 1H). LRMS for C₁₄H₅Cl₄N₃O₄ [M+MeCN+H⁺] m/z=461.

c. Synthesis of Reduction Reaction

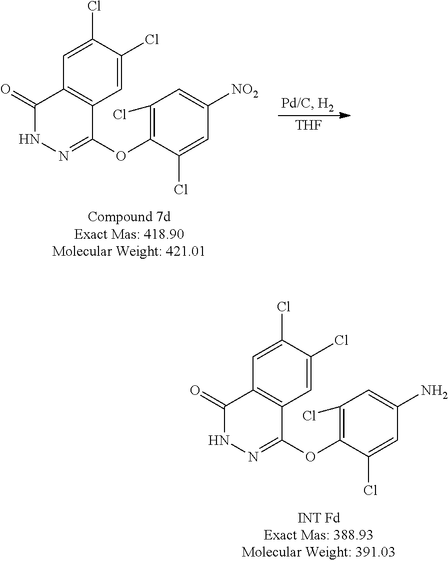 [SMILES: O=c1[nH]nc(Oc2c(Cl)cc([N+](=O)[O-])cc2Cl)c2cc(Cl)c(Cl)cc12 ; "Pd/C, H2" ; Nc1cc(Cl)c(Oc2n[nH]c(=O)c3cc(Cl)c(Cl)cc23)c(Cl)c1 ; "THF"]

Palladium on carbon (5%, 180 mg) was added to the solution of Compound 7d (300 mg, 0.71 mmol) in THF (40 mL). The solution was purged with nitrogen twice and then stirred under hydrogen atmosphere at room temperature for 20 hours. The mixture was filtered through a celite pad, and the filtrate was concentrated to dryness to afford crude solid, which was further purified by column chromatography (2→10% MeOH/DCM) to afford INT Fd as a pale yellow (41.6 mg, 15%).

INT Fd: ¹H NMR (600 MHz, DMSO-d₆) δ 12.18 (s, 1H), 8.41 (d, J=3.1 Hz, 2H), 6.71 (s, 2H), 5.68 (s, 2H). LRMS for C₁₄H₇Cl₄N₃O₂ [M+H⁺] m/z=390.

d. Synthesis of Condensation Reaction

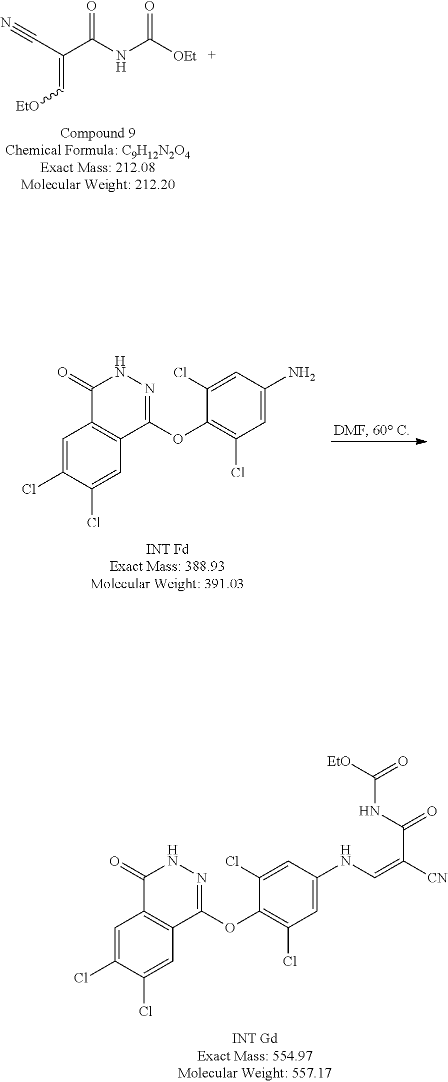

Compound 9 (58.3 mg, 0.27 mmol) was added to a solution of INT Fd (35.0 mg, 0.09 mmol in DMF (1 mL). The mixture was warmed to 60° C. for 5 hours. The mixture was cooled, and the resulting suspension was filtered. The resulting suspension was cooled, and water was added (2.0 mL) and filtered. The filter cake was washed with water then dried in a vacuum oven to afford INT Gd as an inseparable E,Z isomers of as an off-white solid (38.5 mg, yield: 77%).

INT G: ¹H NMR (600 MHz, DMSO-d₆) δ 12.25 (s, 1H), 10.61 (d, J=13.4 Hz, 1H), 10.47 (s, 1H), 8.46 (s, 1H), 8.42 (s, 1H), 7.94 (d, J=10.1 Hz, 1H), 7.80 (s, 2H), 4.15 (q, J=7.1 Hz, 2H), 1.25 (t, J=7.1 Hz, 3H). LRMS for C₂₁H₁₃C₁₄N₅O₅ [M+H⁺] m/z=556.

e. Synthesis of Cyclization Reaction

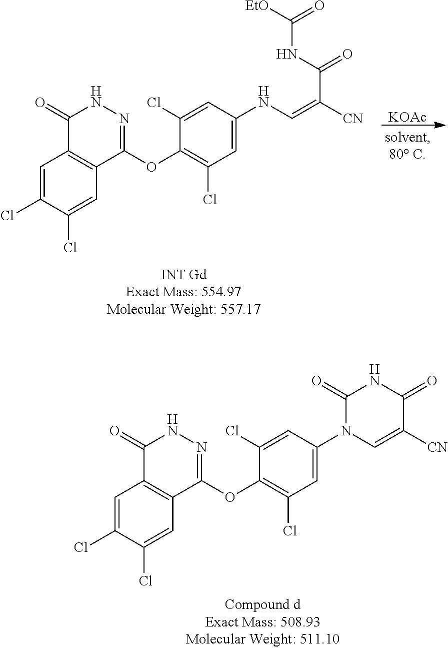 [SMILES: CCOC(=O)NC(=O)/C(C#N)=C\Nc1cc(Cl)c(Oc2n[nH]c(=O)c3cc(Cl)c(Cl)cc23)c(Cl)c1 ; N#Cc1cn(-c2cc(Cl)c(Oc3n[nH]c(=O)c4cc(Cl)c(Cl)cc34)c(Cl)c2)c(=O)[nH]c1=O ; "KOAc"]

INT Gd (30.67 mg, 0.06 mmol) was added to a solution containing potassium acetate (11.2 mg, 0.141 mmol), MeOH (1.0 mL), and water (1.0 mL). The mixture was purged with N₂, the flask was sealed, and heated to 80° C. for 5 hours. The resulting suspension was cooled, and water was added (2.0 mL) and filtered. The filter cake was washed with water then dried in a vacuum oven to afford Compound d as an off-white solid (16.4 mg, yield: 60%).

Compound d: ¹H NMR (600 MHz, DMSO-d₆) δ 12.32 (s, 1H), 12.29 (s, 1H), 8.92 (s, 1H), 8.49 (s, 1H), 8.43 (s, 1H), 7.92 (s, 2H). LRMS for C₁₉H₇C₁₄N₅O₄ [M+ACN+H⁺] m/z=551.

### Synthesis of 1-(3,5-dichloro-4-((6,7-difluoro-4-oxo-3,4-dihydrophthalazin-1-yl)oxy)phenyl)-2,4-dioxo-1,2,3,4-tetrahydropyrimidine-5-carbonitrile (Compound e)

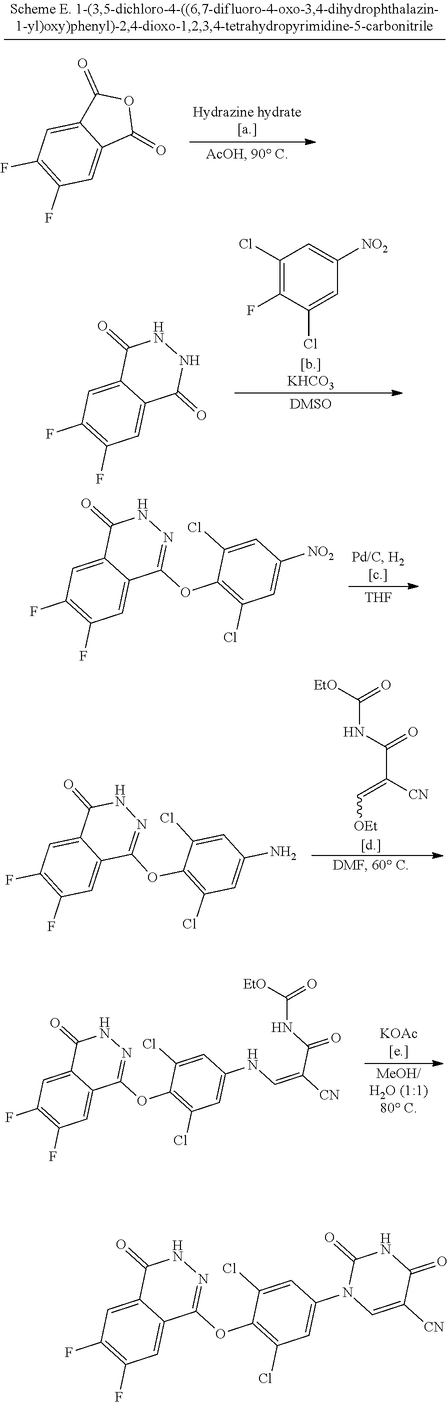

a. Synthesis of Hydrazine Reaction

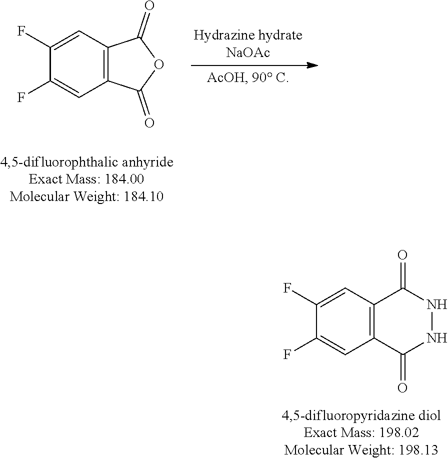 [SMILES: O=C1OC(=O)c2cc(F)c(F)cc21 ; O=c1[nH][nH]c(=O)c2cc(F)c(F)cc12]

Sodium acetate (0.53 g, 7.0 mmol) was added to a solution of 4,5-difluorophthalic anhydride (945 mg, 5.13 mmol) in acetic acid (8.0 mL) and stirred. Then hydrazine hydrate (0.32 mL, 7.0 mmol) was added dropwise and stirred for 15 mins. The mixture was warmed to 80-90° C. for 18 hours. The solid was filtered and washed with water and dried under vacuum for 24 hours to afford Compound 4e as a white solid (970 mg, yield: 91%).

Compound 4e: ¹H NMR (600 MHz, DMSO-d₆) δ 11.76 (s, 2H), 8.01 (s, 2H). LRMS for C₈H₄F₂N₂O₂ [M+H⁺] m/z=199.

b. Synthesis of SnAr Reaction

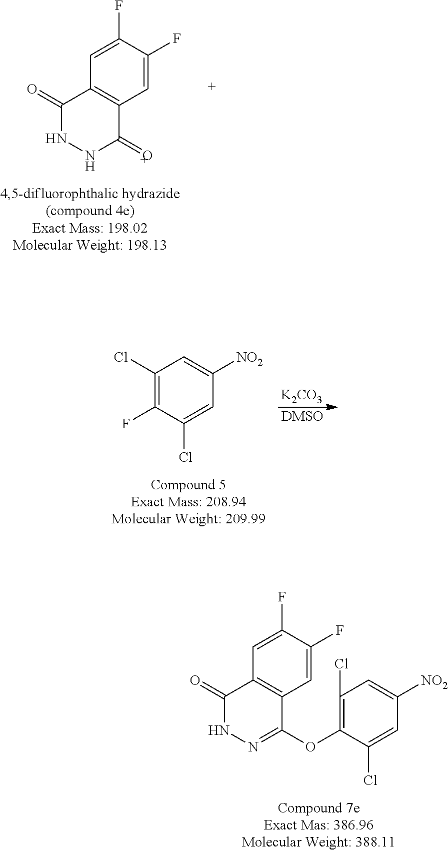

Potassium bicarbonate (313 mg, 3.13 mmol) was added to the solution of 3,6-difluorophthalic hydrazide (Compound 4e, 511 mg, 2.58 mmol) and Compound 5 (313 mg, 1.49 mmol) in DMSO (60 mL) and the mixture was stirred at room temperature for 24 hours before the reaction was quenched by water (60 mL). The resulting suspension was filtered. The filter cake was washed with water (20 mL) and dried under vacuum for 24 hours to afford crude compound 7e, which was further purified by column chromatography (2→10% MeOH/DCM) Compound 7e as an off-white solid (54 mg, yield: 10%).

Compound 7e: ¹H NMR (600 MHz, DMSO-d₆) δ 12.63 (s, 1H), 8.59 (s, 2H), 8.31-8.28 (m, 1H), 8.09 (dd, J=10.1, 7.3 Hz, 1H). LRMS for C₁₄H₅Cl₂F₂N₃O₄ [M+MeCN+H⁺] m/z=429.

c. Synthesis of Reduction Reaction

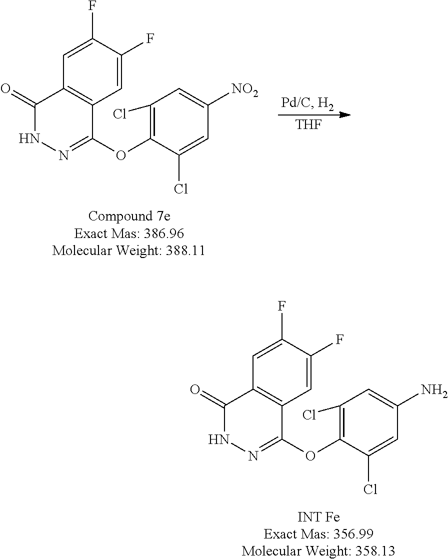 [SMILES: O=c1[nH]nc(Oc2c(Cl)cc([N+](=O)[O-])cc2Cl)c2cc(F)c(F)cc12 ; "Pd/C, H2" ; Nc1cc(Cl)c(Oc2n[nH]c(=O)c3cc(F)c(F)cc23)c(Cl)c1 ; "THF"]

Palladium on carbon (5%, 112 mg) was added to the solution of Compound 7e (120 mg, 0.31 mmol) in THF (5 mL). The solution was purged with nitrogen twice and then stirred under hydrogen atmosphere at room temperature for 2.5 hours. The mixture was filtered through a celite pad, and the filtrate was concentrated to dryness to afford INT Fe as a light tan solid (53.2 mg, 48%).

INT Fe: ¹H NMR (600 MHz, DMSO-d₆) δ 12.13 (s, 1H), 8.25 (ddd, J=25.2, 10.1, 7.4 Hz, 2H), 6.88 (s, 1H), 6.71 (s, 2H), 5.68 (s, 1H). LRMS for C₁₄H₇Cl₂F₂N₃O₂ [M+H⁺] m/z=358.

d. Synthesis of Condensation Reaction

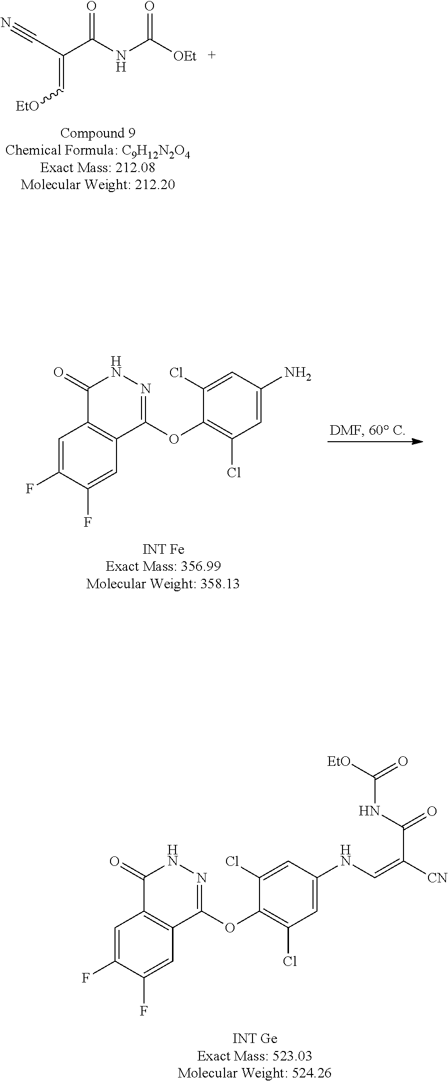

Compound 9 (92.5 mg, 0.43 mmol) was added to a solution of INT Fe (52.2 mg, 0.15 mmol) in DMF (1.2 mL). The mixture was warmed to 60° C. for 3 hours. The mixture was cooled, and water (2 mL) was added, and the resulting suspension was filtered. The filter cake was washed with water (1 mL) and dried under vacuum for 24 hours to afford a mixture of inseparable E,Z isomers of INT Ge as an off-white solid (50.0 mg, yield: 71%).

INT Ge: ¹H NMR (600 MHz, DMSO-d₆, major isomer) δ 12.20 (s, 1H), 10.61 (d, J=13.4 Hz, 1H), 10.47 (s, 1H), 8.49 (d, J=13.4 Hz, 1H), 7.80 (s, 2H), 4.15 (q, J=7.1 Hz, 2H), 2.18 (s, 1H), 1.25 (t, J=7.1 Hz, 3H). LRMS for C₂₁H₁₃Cl₂F₂N₅O₅ [M+H⁺] m/z=524.

e. Synthesis of Cyclization Reaction

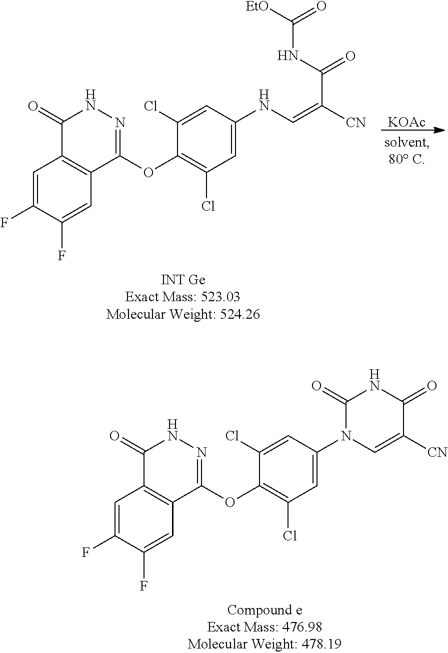 [SMILES: CCOC(=O)NC(=O)/C(C#N)=C\Nc1cc(Cl)c(Oc2n[nH]c(=O)c3cc(F)c(F)cc23)c(Cl)c1 ; N#Cc1cn(-c2cc(Cl)c(Oc3n[nH]c(=O)c4cc(F)c(F)cc34)c(Cl)c2)c(=O)[nH]c1=O ; "KOAc"]

INT Ge (25.0 mg, 0.048 mmol) was added to a solution containing potassium acetate (15.5 mg, 0.158 mmol), MeOH (1.0 mL), and water (1.0 mL). The mixture was purged with N₂, the flask was sealed, and heated to 80° C. for 8 hours. The resulting suspension was cooled, and water was added (2.0 mL) and filtered. The filter cake was washed with water then dried in a vacuum oven to afford Compound e as a tan solid (12.6 mg, yield: 82%).

Compound e: ¹H NMR (600 MHz, DMSO-d₆) δ 12.32 (s, 1H), 12.25 (s, 1H), 8.92 (s, 1H), 8.36 (d, J=9.9 Hz, 1H), 8.26 (d, J=10.0 Hz, 1H), 7.92 (s, 2H). LRMS for C₁₉H₇Cl₂F₂N₅O₄ [M+MeCN+H⁺] m/z=519.

### Synthesis of 1-(3,5-dichloro-4-((5,8-difluoro-4-oxo-3,4-dihydrophthalazin-1-yl)oxy)phenyl)-2,4-dioxo-1,2,3,4-tetrahydropyrimidine-5-carbonitrile (Compound f)

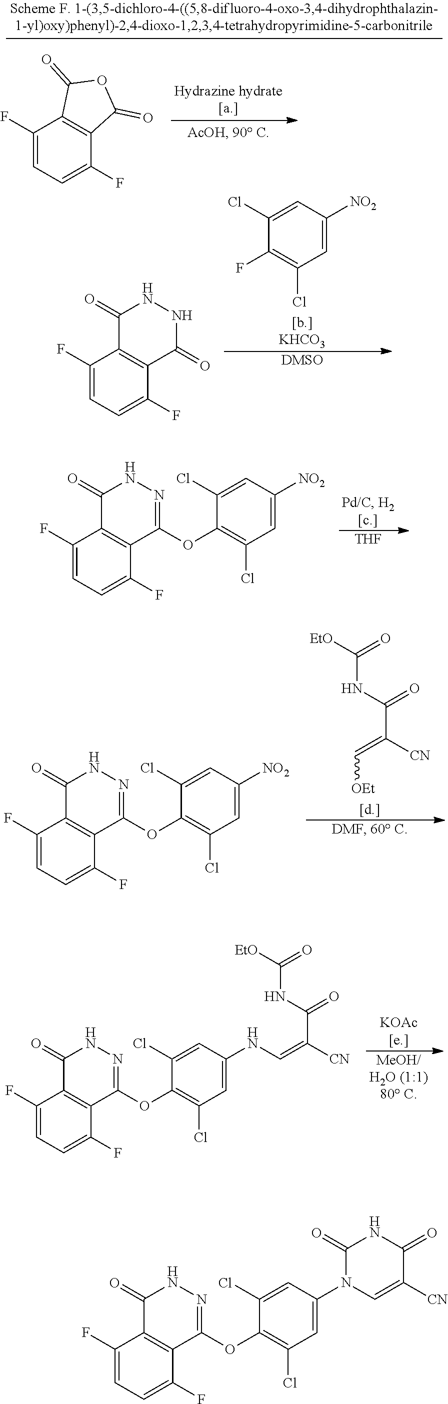

a. Synthesis of Hydrazine Reaction

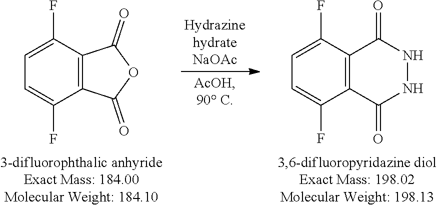 [SMILES: O=C1OC(=O)c2c(F)ccc(F)c21 ; O=c1[nH][nH]c(=O)c2c(F)ccc(F)c12]

Sodium acetate (544 mg, 6.63 mmol) was added to a solution of 3,6-difluorophthalic anhydride (947 mg, 5.14 mmol) in acetic acid (3 mL) and stirred. Then hydrazine hydrate (0.175 mL, 3.60 mmol) was added dropwise and stirred for 15 mins. The mixture was warmed to 80-90° C. for 18 hours. The solid was filtered and washed with water and dried under vacuum for 24 hours to afford Compound 4f as a white solid (1.09, yield: 91.8%).

Compound 4f: ¹H NMR (600 MHz, DMSO-d₆) δ 11.57 (d, J=157.7 Hz, 2H), 7.69 (s, 2H). LRMS for C₈H₄F₂N₂O₂ [M+H⁺] m/z=199.

b. Synthesis of SnAr Reaction

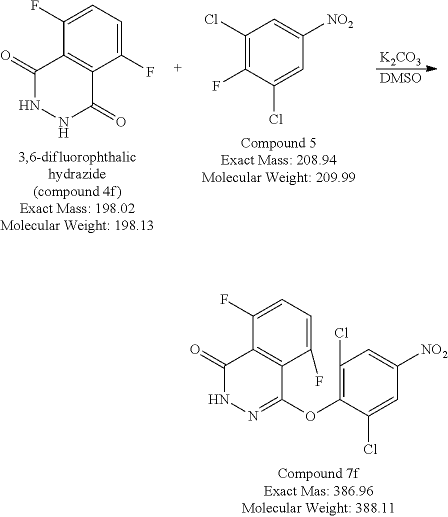

Potassium bicarbonate (240 mg, 2.40 mmol) was added to the solution of 3,6-difluorophthalic hydrazide (Compound 4f, 400 mg, 2.02 mmol) and Compound 5 (160 mg, 0.76 mmol) in DMSO 100 mL) and the mixture was stirred at room temperature for 24 hours before the reaction was quenched by water (60 mL). The resulting suspension was filtered. The filter cake was washed with water (20 mL) and dried under vacuum for 24 hours to afford crude compound 7f, which was further purified by column chromatography (2→10% MeOH/DCM) Compound 7f as a yellow tan solid (101 g, yield: 42%).

Compound 7f: ¹H NMR (600 MHz, DMSO-d₆) δ 12.17 (s, 1H), 8.55 (s, 2H), 8.05-7.79 (m, 2H). LRMS for C₁₄H₅Cl₂F₂N₃O₄ [M+MeCN+H⁺] m/z=429

c. Synthesis of Reduction Reaction

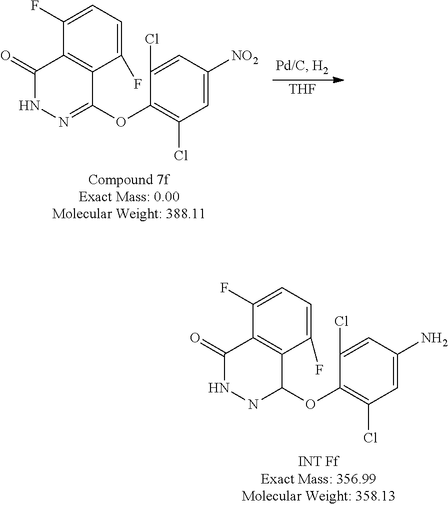 [SMILES: O=c1[nH]nc(Oc2c(Cl)cc([N+](=O)[O-])cc2Cl)c2c(F)ccc(F)c12 ; Nc1cc(Cl)c(OC2NNC(=O)c3c(F)ccc(F)c32)c(Cl)c1 ; "THF"]

Palladium on carbon (5%, 9.6 mg) was added to the solution of Compound 7f (89.4 mg, 0.23 mmol) in THF (10 mL). The solution was purged with nitrogen twice and then stirred under hydrogen atmosphere at room temperature for 6 hours. The mixture was filtered through a celite pad, and the filtrate was concentrated to dryness to afford crude INT Ff, which was further purified by column chromatography (0->4% MeOH/DCM as a light brown solid (32.3 mg, 39%).

INT Ff: ¹H NMR (600 MHz, DMSO-d₆) δ 12.01 (s, 1H), 7.95-7.88 (m, 1H), 7.80 (td, J=9.9, 3.6 Hz, 1H), 6.69 (s, 2H), 5.64 (s, 2H). LRMS for C₁₄H₇Cl₂F₂N₃O₂ [M+H⁺] m/z=358.

d. Synthesis of Condensation Reaction

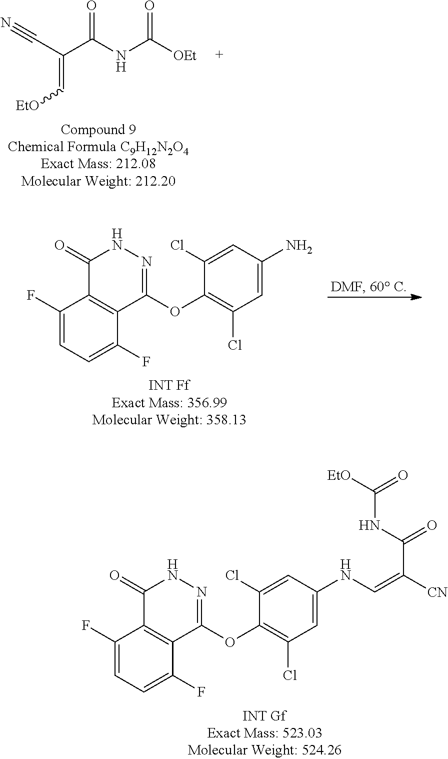 [SMILES: CCOC=C(C#N)C(=O)NC(=O)OCC ; Nc1cc(Cl)c(Oc2n[nH]c(=O)c3c(F)ccc(F)c23)c(Cl)c1 ; "DMF, 60° C." ; CCOC(=O)NC(=O)/C(C#N)=C\Nc1cc(Cl)c(Oc2n[nH]c(=O)c3c(F)ccc(F)c23)c(Cl)c1]

Compound 9 (60.8 mg, 0.29 mmol) was added to a solution of INT Ff (30.6 mg, 0.09 mmol) in DMF (1 mL). The mixture was warmed to 60° C. for 5 hours. The mixture was cooled, water was added (2 mL) and the resulting suspension was filtered. The filter cake was washed with water (2 mL) and dried under vacuum for 24 hours to afford a mixture of inseparable E,Z isomers of INT Gf as an off-white solid (36.7 mg, yield: 78%).

INT Gf: ¹H NMR (600 MHz, DMSO-d₆, major isomer) δ 12.09 (s, 1H), 10.60 (d, J=13.4 Hz, 1H), 10.47 (s, 1H), 8.48 (d, J=13.4 Hz, 1H), 7.97-7.94 (m, 1H), 7.85-7.82 (m, 1H), 7.79 (s, 2H), 4.15 (q, J=7.1 Hz, 2H), 1.25 (t, J=7.1 Hz, 3H). LRMS for C₂₁H₁₃Cl₂F₂N₅O₅ [M+H⁺]m/z=524.

e. Synthesis of Cyclization Reaction

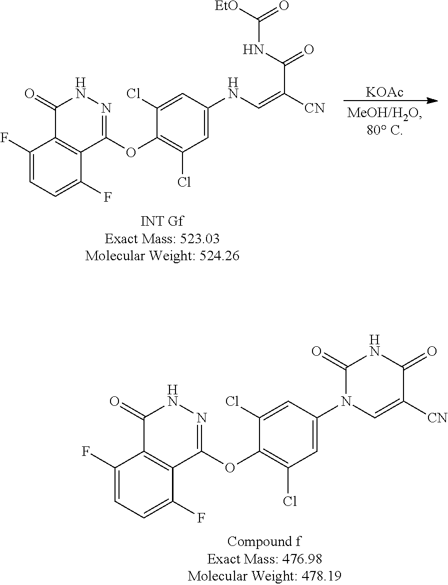 [SMILES: CCOC(=O)NC(=O)/C(C#N)=C\Nc1cc(Cl)c(Oc2n[nH]c(=O)c3c(F)ccc(F)c23)c(Cl)c1 ; N#Cc1cn(-c2cc(Cl)c(Oc3n[nH]c(=O)c4c(F)ccc(F)c34)c(Cl)c2)c(=O)[nH]c1=O ; "KOAc"]

INT Gf (31.9 mg, 0.06 mmol) was added to a solution containing potassium acetate (14.0 mg, 0.14 mmol), MeOH (1.0 mL), and water (1.0 mL). The mixture was purged with N₂, the flask was sealed, and heated to 80° C. for 4 hours. The resulting suspension was cooled, and water was added (2.0 mL) and filtered. The filter cake was washed with water then dried in a vacuum oven to afford Compound f as an off-white solid (23.0 mg, yield: 94%).

Compound f: ¹H NMR (600 MHz, DMSO-d₆) δ 12.31 (s, 1H), 12.14 (s, 1H), 8.90 (s, 1H), 7.99-7.96 (m, 1H), 7.91 (s, 2H), 7.86-7.83 (m, 1H). LRMS for C₁₉H₇Cl₂F₂N₅O₄ [M+H⁺] m/z=478.

### Synthesis of 1-(3,5-dichloro-4-((5-methoxy-4-oxo-3,4-dihydrophthalazin-1-yl)oxy)phenyl)-2,4-dioxo-1,2,3,4-tetrahydropyrimidine-5-carbonitrile (Compound h)

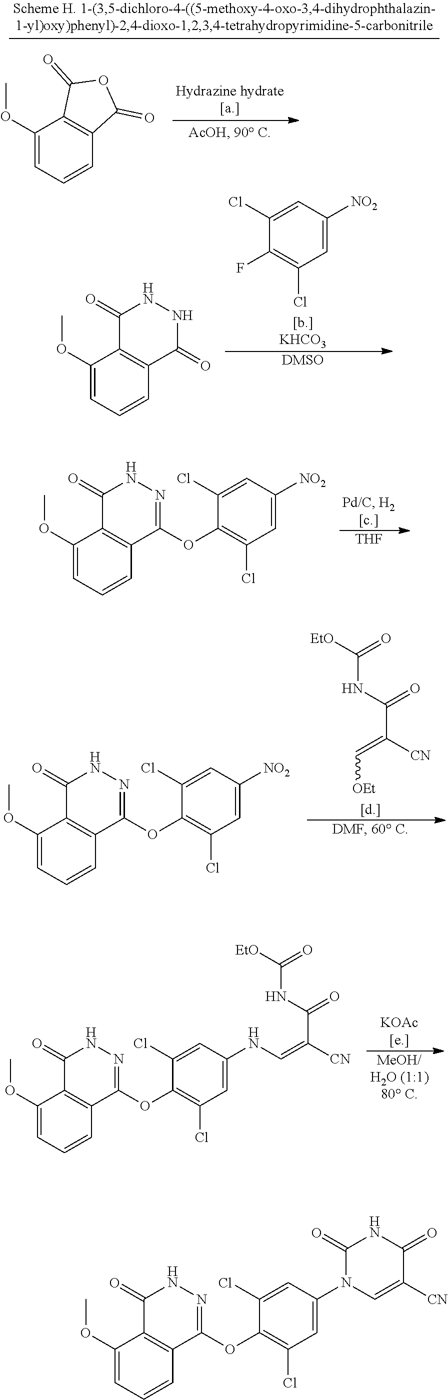

a. Synthesis of Hydrazine Reaction

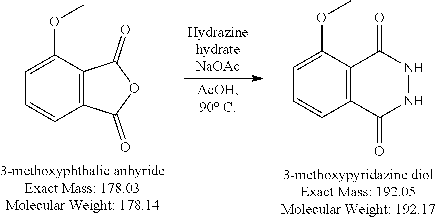

Sodium acetate (300.1 mg, 3.66 mmol) was added to a solution of 3-methyloxyphthalic anhydride (489.8 mg, 2.75 mmol) in acetic acid (2.4 mL) and stirred. Then hydrazine hydrate (0.16 mL, 3.40 mmol) was added dropwise and stirred for 15 mins. The mixture was warmed to 80-90° C. for 18 hours. The solid was filtered and washed with water and dried under vacuum for 24 hours to afford Compound 4h as a white solid (417 mg, yield: 79%).

Compound 4h: ¹H NMR (600 MHz, DMSO-d₆) δ 7.79 (t, J=8.0 Hz, 1H), 7.70-7.55 (m, 1H), 7.42 (d, J=8.4 Hz, 1H), 3.90 (s, 3H). LRMS for C₉H₈N₂O₃ [M+H⁺] m/z=192.

b. Synthesis of SnAr Reaction

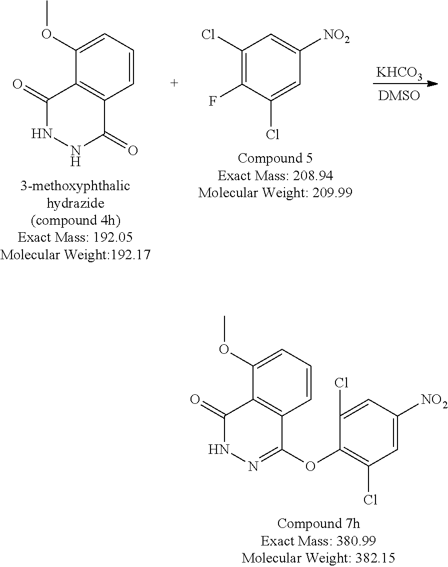

Potassium bicarbonate (313 mg, 3.13 mmol) was added to the solution of 3-methyoxyphthalic hydrazide (Compound 4 h, 400 mg, 2.08 mmol) and Compound 5 (277 g, 1.32 mmol) in DMSO (30 mL) and the mixture was stirred at room temperature for 24 hours before the reaction was quenched by water (30 mL). The resulting suspension was filtered. The filter cake was washed with water (20 mL) and dried under vacuum for 24 hours to afford crude compound 7h (496 mg), which was further purified by slurry in ethyl acetate to afford Compound 7h as a pale-yellow solid (370 mg, yield: 74%).

Compound 7h: ¹H NMR (600 MHz, DMSO-d₆) δ 11.69 (s, 1H), 8.54 (s, 2H), 7.99 (t, J=8.1 Hz, 1H), 7.72 (d, J=7.9 Hz, 1H), 7.55 (d, J=8.5 Hz, 1H), 3.94 (s, 3H). LRMS for C₁₅H₉Cl₂N₃O₅ [M+H⁺] m/z=382.

c. Synthesis of Reduction Reaction

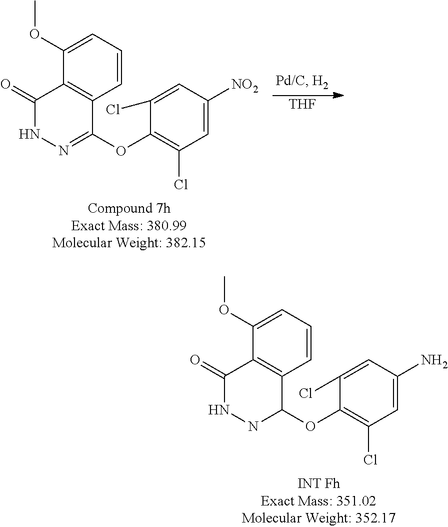 [SMILES: COc1cccc2c(Oc3c(Cl)cc([N+](=O)[O-])cc3Cl)n[nH]c(=O)c12 ; COc1cccc2c1C(=O)NNC2Oc1c(Cl)cc(N)cc1Cl ; "THF"]

Palladium on carbon (5%, 0.02 g) was added to the solution of Compound 7h (251 mg, 0.66 mmol) in THF (2 mL). The solution was purged with nitrogen twice and then stirred under hydrogen atmosphere at room temperature for 5 hours. The mixture was filtered through a celite pad, and the filtrate was concentrated to dryness to afford crude INT Fh. The crude material was purified by column chromatography (2→20% DCM:MeOH) to afford pure INT Fh as a pale yellow solid (61.1 mg, 26.2%).

INT Fh: ¹H NMR (600 MHz, DMSO-d₆) δ 11.53 (s, 1H), 7.93 (t, J=8.1 Hz, 1H), 7.68 (d, J=6.9 Hz, 1H), 7.49 (dd, J=8.5, 1.0 Hz, 1H), 6.69 (s, 2H), 5.62 (s, 2H), 3.92 (s, 3H). LRMS for C₁₅H₁₁C₂N₃O₃ [M+H⁺] m/z=352).

d. Synthesis of Condensation Reaction

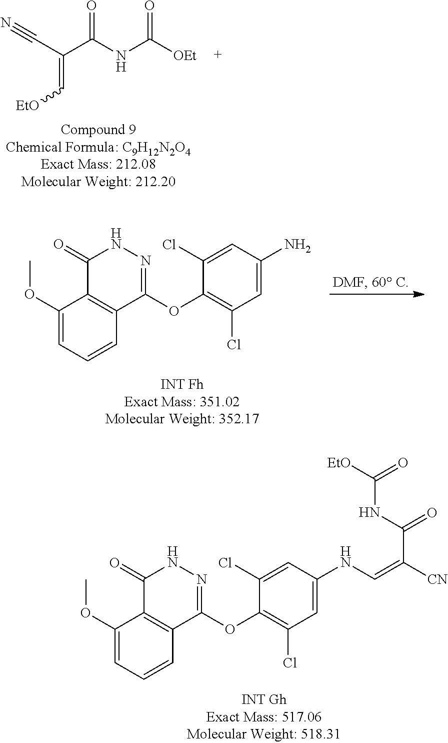 [SMILES: CCOC=C(C#N)C(=O)NC(=O)OCC ; COc1cccc2c(Oc3c(Cl)cc(N)cc3Cl)n[nH]c(=O)c12 ; CCOC(=O)NC(=O)/C(C#N)=C\Nc1cc(Cl)c(Oc2n[nH]c(=O)c3c(OC)cccc23)c(Cl)c1]

Compound 9 (93 mg, 0.44 mmol) was added to a solution of INT Fh (50 mg, 0.14 mmol) in DMF (1 mL). The mixture was warmed to 80° C. for 5 hours. The mixture was cooled, water was added (2 mL) and the resulting suspension was filtered. The filter cake was washed with water (2 mL) and dried under vacuum for 24 hours to afford a mixture of inseparable E,Z isomers of INT Gh as an off-white solid (74.9 mg, yield: 14%).

INT Gh: ¹H NMR (600 MHz, DMSO-d₆, major isomer) δ 11.61 (s, 1H), 10.60 (d, J=13.5 Hz, 1H), 10.47 (s, 1H), 8.48 (d, J=13.4 Hz, 1H), 7.97 (d, J=8.3 Hz, 1H), 7.78 (s, 2H), 7.71 (d, J=8.9 Hz, 1H), 7.53 (d, J=9.6 Hz, 1H), 4.15 (q, J=6.7 Hz, 2H), 3.93 (s, 3H), 1.25 (t, J=7.1 Hz, 3H). LRMS for C₂₂H₁₇Cl₂N₅O₆ [M+H⁺] m/z=518.

e. Synthesis of Cyclization Reaction

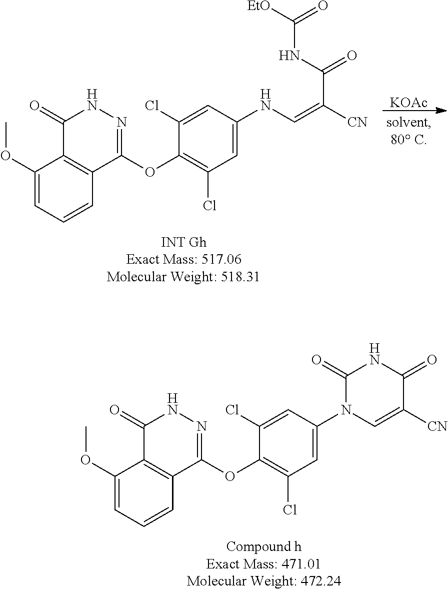 [SMILES: CCOC(=O)NC(=O)/C(C#N)=C\Nc1cc(Cl)c(Oc2n[nH]c(=O)c3c(OC)cccc23)c(Cl)c1 ; COc1cccc2c(Oc3c(Cl)cc(-n4cc(C#N)c(=O)[nH]c4=O)cc3Cl)n[nH]c(=O)c12 ; "KOAc"]

INT Gh (41.0 mg, 0.08 mmol) was added to a solution containing potassium acetate (15.8 mg, 0.16 mmol), MeOH (1.0 mL), and Water (1.0 mL). The mixture was purged with N₂, the flask was sealed, and heated to 80° C. for 6 h. The resulting suspension was cooled and filtered. The filter cake was washed with water to provide purified Compound h as an off-white solid (30.2 mg, yield: 82.9%).

Compound h: ¹H NMR (600 MHz, DMSO-d₆) δ 12.31 (s, 1H), 11.65 (s, 1H), 8.91 (s, 1H), 7.99 (t, J=8.2 Hz, 1H), 7.90 (s, 2H), 7.73 (d, J=7.9 Hz, 1H), 7.55 (d, J=7.7 Hz, 1H), 3.94 (s, 3H). LRMS for C₂₀H₁₁Cl₂N₅O₅ [M+H⁺] m/z=472.

### Synthesis of 1-(3,5-dichloro-4-((4-oxo-3,4-dihydrophthalazin-1-yl)oxy)phenyl)-2,4-dioxo-1,2,3,4-tetrahydropyrimidine-5-carboxamide (Compound i)

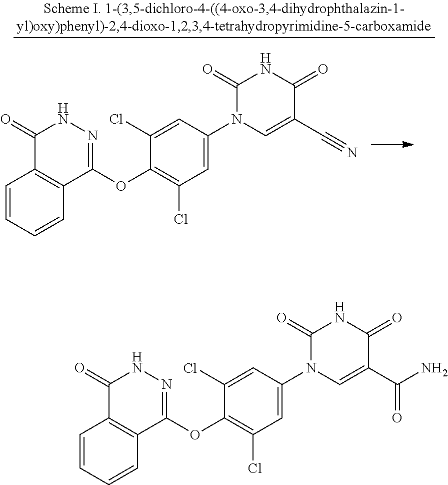 [SMILES: N#Cc1cn(-c2cc(Cl)c(Oc3n[nH]c(=O)c4ccccc34)c(Cl)c2)c(=O)[nH]c1=O ; NC(=O)c1cn(-c2cc(Cl)c(Oc3n[nH]c(=O)c4ccccc34)c(Cl)c2)c(=O)[nH]c1=O]

a. Functional Group Conversion

1-(3,5-dichloro-4-((4-oxo-3,4-dihydrophthalazin-1-yl)oxy)phenyl)-2,4-dioxo-1,2,3,4-tetrahydropyrimidine-5-carbonitrile (20 mg, 0.05 mol) and sulfuric acid (0.5 mL) were added to a 3 mL vial and warmed to 75-80° C. for 5 mins. The vial was cooled to room temperature, then the solution was added to water (2 mL). The solid was filtered and washed with water and dried under vacuum for 24 hours to afford Compound i as a white solid (20.1 mg, yield: 98%).

Compound i: ¹H NMR (600 MHz, DMSO-d₆) δ 12.12 (s, 1H), 12.03 (s, 1H), 8.37 (s, 1H), 8.32 (d, J=9.9 Hz, 1H), 8.25 (d, J=6.8 Hz, 1H), 8.14 (s, 1H), 8.11-8.08 (m, 1H), 8.03 (t, J=8.3 Hz, 1H), 7.94 (s, 2H), 7.66 (s, 1H). LRMS for C₁₉H₁₁C₂N₅O₅ [M+MeCN+H⁺] m/z=501.

### Synthesis of 1-(3,5-dichloro-4-((4-oxo-3,4-dihydrophthalazin-1-yl)oxy)phenyl)-2,4-dioxo-1,2,3,4-tetrahydropyrimidine-5-carboxylic acid (Compound j)

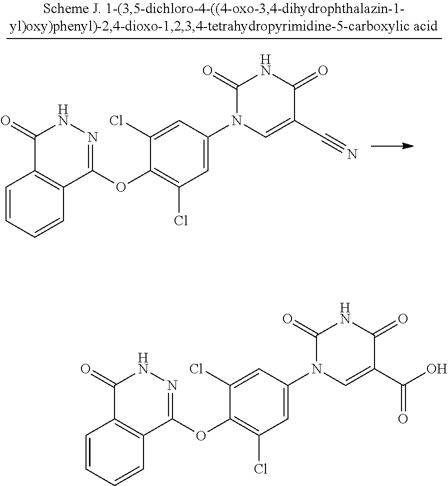 [SMILES: N#Cc1cn(-c2cc(Cl)c(Oc3n[nH]c(=O)c4ccccc34)c(Cl)c2)c(=O)[nH]c1=O ; O=C(O)c1cn(-c2cc(Cl)c(Oc3n[nH]c(=O)c4ccccc34)c(Cl)c2)c(=O)[nH]c1=O]

a. Functional Group Conversion

1-(3,5-dichloro-4-((4-oxo-3,4-dihydrophthalazin-1-yl)oxy)phenyl)-2,4-dioxo-1,2,3,4-tetrahydropyrimidine-5-carbonitrile (51.2 mg, 0.05 mol), acetic acid (4 mL), sulfuric acid (2 mL) and water (2 mL) were added to a 20 mL vial and warmed to 100° C. for 9 days. The vial was cooled to room temperature. The solid was filtered and washed with water and dried under vacuum for 24 hours to afford Compound j as a white solid (45.1 mg, yield: 99%).

Compound j: ¹H NMR (600 MHz, DMSO-d₆) δ 12.25 (s, 1H), 12.04 (s, 1H), 8.56 (s, 1H), 8.32 (d, J=7.2 Hz, 1H), 8.25 (d, J=7.4 Hz, 1H), 8.11-8.07 (m, 1H), 8.04 (dd, J=7.6, 1.5 Hz, 1H), 7.94 (s, 2H). LRMS for C₁₉H₁₀Cl₂N₄O₆ [M+MeCN+H⁺] m/z=502.

### Example 2—Biological Assay

Thyroid receptor beta and thyroid receptor alpha coactivator recruitment assays were performed using LanthaScreen™ TR-FRET TR alpha or beta Coactivator Assay Kits (ThermoFIsher). The control agonist 3,3′,5-Triiodo-L-thyronine sodium salt, T3 (SIGMA) was used to validate the assay. Test compound and/or vehicle was incubated with the TR alpha LBD-GST or TR beta LBD-GST and coactivator peptide (Fluorescein-SRC2-2 (LKEKHKILHRLLQDSSSPV) (SEQ ID NO: 1)) for 30 minutes at RT. Determination of the amount of complex formed was read spectrofluorimetrically (excitation: 337 nm, emission: 520/490 nm) on a PHERAstar FS instrument (BMG). Test compound-induced increases in fluorescence greater than 50 percent relative to the T3 response were used to calculate EC₅₀ values. Compounds were screened at 30, 10, 3, 1, 0.3, 0.1, and 0.03 μM.

TABLE 1

| | | | Concentration | | |
|---|---|---|---|---|---|
| Compound | | | of Test | Units of | Agonist |
| ID | Ascii Assay Name | Solvent | Compound | Concentration | Response |
| g | Thyroid Receptor | 1% | 30 | mcM | 18.6 |
| | Alpha Coactivator - | DMSO | | | |
| | Agonist | | | | |
| g | Thyroid Receptor | 1% | 10 | mcM | 19.5 |
| | Alpha Coactivator - | DMSO | | | |
| | Agonist | | | | |
| g | Thyroid Receptor | 1% | 3 | mcM | 19.2 |
| | Alpha Coactivator - | DMSO | | | |
| | Agonist | | | | |
| g | Thyroid Receptor | 1% | 1 | mcM | 12.7 |
| | Alpha Coactivator - | DMSO | | | |
| | Agonist | | | | |
| g | Thyroid Receptor | 1% | 0.3 | mcM | 3.9 |
| | Alpha Coactivator - | DMSO | | | |
| | Agonist | | | | |
| g | Thyroid Receptor | 1% | 0.1 | mcM | 2.2 |
| | Alpha Coactivator - | DMSO | | | |
| | Agonist | | | | |
| g | Thyroid Receptor | 1% | 0.03 | mcM | 1.4 |
| | Alpha Coactivator - | DMSO | | | |
| | Agonist | | | | |
| g | Thyroid Receptor | 1% | 10 | nM | 1.1 |
| | Alpha Coactivator - | DMSO | | | |
| | Agonist | | | | |
| g | Thyroid Receptor Beta | 1% | 30 | mcM | 31.8 |
| | Coactivator - Agonist | DMSO | | | |
| g | Thyroid Receptor Beta | 1% | 10 | mcM | 32.6 |
| | Coactivator - Agonist | DMSO | | | |
| g | Thyroid Receptor Beta | 1% | 3 | mcM | 31.1 |
| | Coactivator - Agonist | DMSO | | | |
| g | Thyroid Receptor Beta | 1% | 1 | mcM | 28.7 |
| | Coactivator - Agonist | DMSO | | | |
| g | Thyroid Receptor Beta | 1% | 0.3 | mcM | 10.5 |
| | Coactivator - Agonist | DMSO | | | |
| g | Thyroid Receptor Beta | 1% | 0.1 | mcM | 0.7 |
| | Coactivator - Agonist | DMSO | | | |
| g | Thyroid Receptor Beta | 1% | 0.03 | mcM | −1.2 |
| | Coactivator - Agonist | DMSO | | | |
| g | Thyroid Receptor Beta | 1% | 10 | nM | −4.5 |
| | Coactivator - Agonist | DMSO | | | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula I: 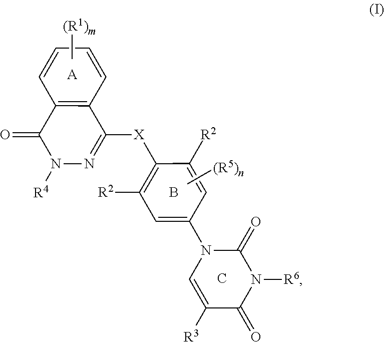
or a pharmaceutically acceptable salt, solvate, or stereoisomer thereof, wherein:
each R¹ is independently halogen, C₁₋₆ alkyl, C₂₋₆ alkenyl, C₂₋₆ alkynyl, C₁₋₆ alkoxy, C₃₋₁₀ carbocyclyl, or C₆₋₁₄ aryl;
each R² is independently halogen, C₁₋₆ alkyl, C₂₋₆ alkenyl, or C₂₋₆ alkynyl;
R³ is halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —R^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
each occurrence of R⁴ and R⁶ is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
each R⁵ is independently halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —N^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u};
m is an integer selected from 0 to 4;
n is an integer selected from 0 to 2;
X is selected from —C(R⁷)₂—, —O—, —NR⁸—, and —S—;
each R⁷ is independently hydrogen, halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, or heteroaryl is optionally substituted with one or more R^{u}; and
R⁸ is hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, or 3- to 10-membered heterocyclyl, wherein the alkyl, haloalkyl, hydroxyalkyl, aminoalkyl, alkenyl, alkynyl, aryl, heteroaryl, carbocyclyl, or heterocyclyl is optionally substituted with one or more R^{u};
wherein:
each R^{u} is independently halogen, —CN, —NO₂, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}R^{d}, —NR^{c}S(═O)₂R^{a}, —NR^{c}S(═O)₂R^{a}, —NR^{c}S(═O)₂OR^{b}, —NR^{c}S(═O)₂NR^{c}R^{d}, —N^{b}C(═O)NR^{c}R^{d}, —N^{b}C(═O)R^{a}, —N^{b}C(═O)OR^{b}, —OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, haloalkyl, hydroxyalkyl, aminoalkyl, alkenyl, alkynyl, carbocyclyl, heterocyclyl, aryl, and heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
two R^{u} are taken together to form an oxo; or
two R^{u}, together with the one or more intervening atoms, form a C₆₋₁₄ aryl, 5- to 14-membered heteroaryl, C₃₋₁₀ carbocyclyl, or 3- to 10-membered heterocyclyl;
each R^{a} is independently C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
each R^{b} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl; and
each R^{c} and R^{d} is independently hydrogen, C₁₋₆ alkyl, C₁₋₆ haloalkyl, C₁₋₆ hydroxyalkyl, C₁₋₆ aminoalkyl, C₂₋₆ alkenyl, C₂₋₆ alkynyl, C₃₋₁₀ carbocyclyl, 3- to 10-membered heterocyclyl, C₆₋₁₄ aryl, or 5- to 14-membered heteroaryl, wherein the alkyl, haloalkyl, hydroxyalkyl, aminoalkyl, alkenyl, alkynyl, carbocyclyl, heterocyclyl, aryl, or heteroaryl is optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl;
or R^{c}, R^{d}, together with the nitrogen atom to which they are attached, form a 3- to 10-membered heterocyclyl optionally substituted with one or more oxo, halogen, —CN, —OH, —OMe, —NH₂, —C(═O)Me, —C(═O)OH, —C(═O)OMe, C₁₋₆ alkyl, or C₁₋₆ haloalkyl.

2. The compound of claim 1, wherein each R¹ is independently halogen, C₁₋₆ alkyl, C₁₋₆ alkoxy, or C₆₋₁₄ aryl.

3. The compound of claim 1, wherein m is 0, 1, or 2.

4. The compound of claim 1, wherein each R² is independently halogen or C₁₋₆ alkyl.

5. The compound of claim 1, wherein each R² is halogen.

6. The compound of claim 1, wherein each R² is chloride.

7. The compound of claim 1, wherein R³ is —CN, —C(═O)OR^{b}, or —C(═O)NR^{c}R^{d}.

8. The compound of claim 1, wherein R³ is —CN.

9. The compound of claim 1, wherein each R^{c} and R^{c} is H.

10. The compound of claim 1, wherein R³ is —C(═O)NH₂.

11. The compound of claim 1, wherein R^{b} is H.

12. The compound of claim 1, wherein R³ is —C(═O)OH.

13. The compound of claim 1, wherein R⁴ is hydrogen.

14. The compound of claim 1, wherein n is 0.

15. The compound of claim 1, wherein R⁶ is hydrogen.

16. The compound of claim 1, wherein X is O.

17. The compound of claim 1, wherein the compound is selected from the compounds described in Table I, and pharmaceutically acceptable salts, stereoisomers, and solvates thereof.

18. A pharmaceutical composition comprising a compound of claim 1, or a pharmaceutically acceptable salt, stereoisomer, or solvate thereof, and a pharmaceutically acceptable excipient.

19. A method of activating thyroid hormone receptor (THR) R in a patient or a biological sample, the method comprising contacting the patient or biological sample with a compound of claim 1, or a pharmaceutically acceptable salt, stereoisomer, or solvate thereof.

20. A method of treating a liver disease or disorder, or a lipid disease or disorder, the method comprising administering to a patient in need thereof a compound of claim 1, or a pharmaceutically acceptable salt, stereoisomer, or solvate thereof.

21. The method of claim 20, wherein the liver disease or disorder is nonalcoholic fatty liver disease (NAFLD), nonalcoholic steatohepatitis (NASH), or fatty liver disease.

22. The method of claim 20, wherein the lipid disease or disorder is dyslipidemia, hyperlipidemia, hypertriglyceridemia, hypercholesterolemia, low high-density lipoprotein (HDL), or high low-density lipoprotein (LDL).

23. The method of claim 22, wherein hypercholesterolemia is heterozygous familial hypercholesterolemia (HeFH) or homozygous familial hypercholesterolemia (HoFH).

24.-31. (canceled)


SEQUENCE LISTING (1 sequence)

SEQ ID NO: 1 - protein, 19 aa, Myotis davidii
LKEKHKILHRLLQDSSSPV